(19) United States
(12) Patent Application Publication
(10) Pub. No.: US 2024/0115521 A1
(43) Pub. Date: Apr. 11, 2024

(54) COMPOUNDS FOR THE TREATMENT OF MYCOBACTERIAL DISEASES

(71) Applicants: Saint Louis University, St. Louis, MO (US); Research Foundation of the City University of New York, New York, NY (US)

(72) Inventors: Getahun ABATE, St. Louis, MO (US); John TAVIS, Kirkwood, MO (US); David W. GRIGGS, Ballwin, MO (US); Ryan MURELLI, Belleville, NJ (US)

(21) Appl. No.: 18/470,317

(22) Filed: Sep. 19, 2023

Related U.S. Application Data
(60) Provisional application No. 63/409,424, filed on Sep. 23, 2022.

(51) Int. Cl. A61K 31/122; A61K 31/136; A61K 31/235; A61K 31/353; A61K 31/402; A61K 31/426; A61K 31/495; A61K 31/496; A61K 31/5375; A61K 31/55; A61K 45/06; A61P 31/04; A61P 31/06

(52) U.S. Cl. CPC A61K 31/122; A61K 31/136; A61K 31/235; A61K 31/353; A61K 31/402; A61K 31/426; A61K 31/495; A61K 31/496; A61K 31/5375; A61K 31/55; A61K 45/06; A61P 31/04; A61P 31/06

(57) ABSTRACT

The present disclosure provides compounds, methods, and compositions which may be used to treat tuberculosis. In some embodiments, these compounds and compositions have a bactericidal property against Mycobacterium tuberculosis (Mtb). Methods of employing such agents are also provided.

This application claims the benefit of U.S. Provisional Application No. 63/409,424, filed Sep. 23, 2022, the contents of which is incorporated herein by reference.

This application was made with Government Support under Grant No. RO1 AI122669 and SC1 GM111158 awarded by the National Institutes of Health. The government has certain rights in the invention.

### BACKGROUND

### I. Field of the Disclosure

The present disclosure relates to the fields of medicine, pharmacology and infectious disease. More particular, the disclosure relates to methods and compositions for treating mycobacterial infections, such as tuberculosis.

### II. Related Art

Mycobacteria are unique groups of bacteria that primarily affect the lungs. Mycobacterium tuberculosis (Mtb) is the cause of tuberculosis (TB) and Mycobacterium avium complex (MAC) is the most common cause of pulmonary nontuberculous mycobacteria (NTM). The standard treatment regimens for new patients with pulmonary TB and MAC are very long, taking 6 months for TB and at least 18 months for MAC. Treatment of pulmonary MAC even for new patients has a failure rate of more than 40%, indicating the dire need to develop new drugs to shorten the treatment regimen and increase efficacy. Available options for the treatment of MAC have not changed in the past >20 years (American Thoracic Society, 1997), but the increasing prevalence in the US and other Western countries over the last decade has led to a renewed interest (Daniel-Wayman et al., 2019).

The emergence and spread of drug-resistant TB, particularly multidrug-resistant (MDR) TB (resistance to at least isoniazid and rifampin, two key first-line drugs), and extensively drug-resistant (XDR) TB (resistance also to important second line drugs such as fluoroquinolones and aminoglycosides) have made TB control extremely challenging. Thus, in 1993, the world health organization (WHO) declared TB an international emergency, making it the first infectious disease to be declared as such. TB remains a high priority area for drug development research. While it is encouraging to note that a regimen containing three new drugs for treatment of MDR-TB was recently approved, resistance to newly developed drugs has already emerged (Ghodousi et al., 2019; Polsfuss et al., 2019). Therefore, finding new drugs for the treatment of drug-resistant TB is crucially important.

### SUMMARY

In some aspects, the present disclosure relates to methods of treating mycobacterial diseases. In one aspect, the present disclosure provides methods of treating an infection of a mycobacteria in a patient comprising administering to the patient a therapeutically effective amount of a compound of the formula:

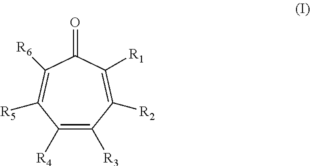

wherein:
R₁ and R₆ are each independently amino, hydroxy, mercapto, alkyl_{(C≤12)}, cycloalkyl_{(C≤12)}, aryl_{(C≤12)}, heteroaryl_{(C≤12)}, heterocycloalkyl_{(C≤12)}, alkoxy_{(C≤12)}, acyloxy_{(C≤12)}, or a substituted version of any of these groups;
R₂ and R₅ are each independently hydrogen or S(O)ₓRₐ, wherein:
   x is 0, 1, or 2; and
   Rₐ is hydrogen, alkyl_{(C≤12)}, cycloalkyl_{(C≤12)}, aryl_{(C≤12)}, aralkyl_{(C≤12)}, heteroaryl_{(C≤12)}, heterocycloalkyl_{(C≤12)}, or a substituted version thereof; and
R₃ and R₄ are each independently hydrogen, alkyl_{(C≤12)}, substituted alkyl_{(C≤12)}, or —C(O)R_{b}, wherein:
   R_{b} is heterocycloalkyl_{(C≤12)}, substituted heterocycloalkyl_{(C≤12)}, heterocycloalkyl_{(C≤12)}-R_{c}, substituted heterocycloalkyl_{(C≤12)}-R_{c}; wherein:
      R_{c} is S(O)_{y}R_{c}′, wherein:
         y is 0, 1, or 2;
         R_{c}′ is hydroxy, alkyl_{(C≤12)}, cycloalkyl_{(C≤12)}, aryl_{(C≤12)}, or a substituted version of any of these groups;
            or a compound of the formula:

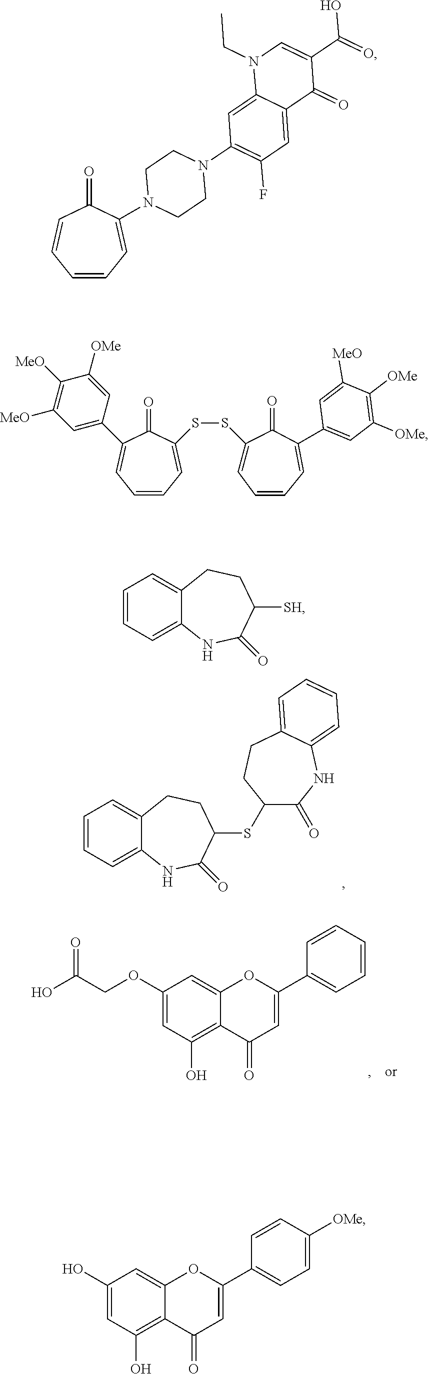

or a pharmaceutically acceptable salt thereof.

In still another aspect, the present disclosure provides methods of killing a mycobacteria comprising contacting the mycobacteria with a compound of the formula:

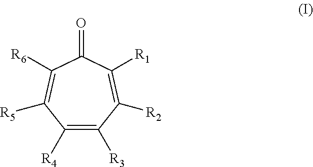

wherein:
R₁ and R₆ are each independently amino, hydroxy, mercapto, alkyl_{(C≤12)}, cycloalkyl_{(C≤12)}, aryl_{(C≤12)}, heteroaryl_{(C≤12)}, heterocycloalkyl_{(C≤12)}, alkoxy_{(C≤}12), acyloxy_{(C≤}12), or a substituted version of any of these groups;
R₂ and R₅ are each independently hydrogen or S(O)ₓRₐ, wherein:
   x is 0, 1, or 2; and
   Rₐ is hydrogen, alkyl_{(C≤12)}, cycloalkyl_{(C≤12)}, aryl_{(C≤12)}, aralkyl_{(C≤12)}, heteroaryl_{(C≤12)}, heterocycloalkyl_{(C≤12)}, or a substituted version thereof; and
R₃ and R₄ are each independently hydrogen, alkyl_{(C≤12)}, substituted alkyl_{(C≤12)}, or —C(O)R_{b}, wherein:
   R_{b} is heterocycloalkyl_{(C≤12)}, substituted heterocycloalkyl_{(C≤12)}, heterocycloalkyl_{(C≤12)}-R_{c}, substituted heterocycloalkyl_{(C≤12)}-R_{c}; wherein:
      R_{c} is S(O)_{y}R_{c}′, wherein:
         y is 0, 1, or 2;
         R_{c}′ is hydroxy, alkyl_{(C≤12)}, cycloalkyl_{(C≤12)}, aryl_{(C≤12)}, or a substituted version of any of these groups;
            or a compound of the formula:

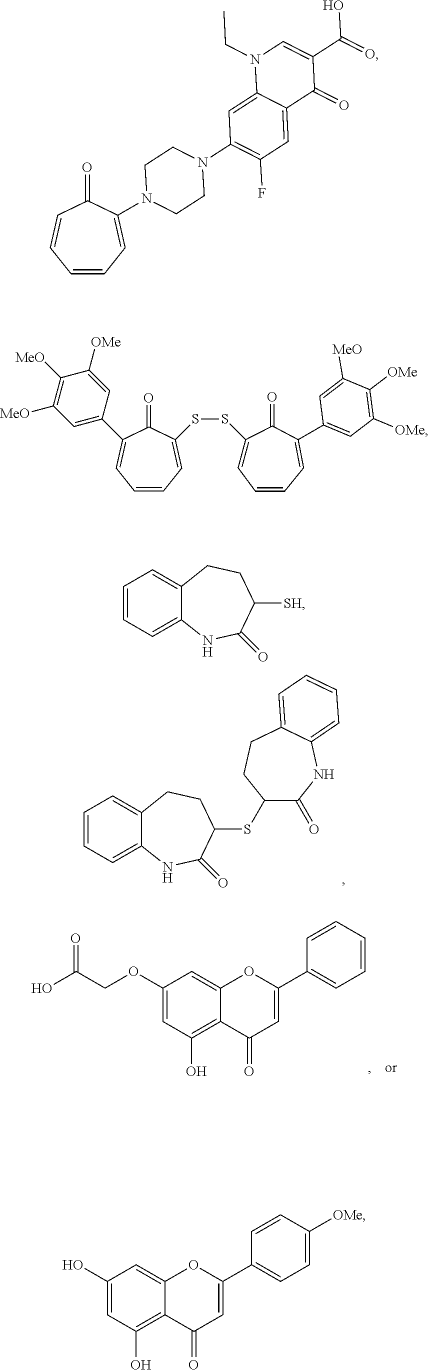

or a pharmaceutically acceptable salt thereof.

In still yet another aspects, the present disclosure provides methods of inhibiting the replication of a mycobacteria comprising contacting the mycobacteria with a compound of the formula:

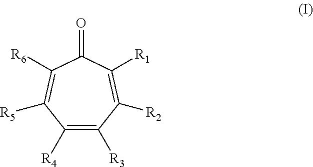

wherein:
R₁ and R₆ are each independently amino, hydroxy, mercapto, alkyl_{(C≤12)}, cycloalkyl_{(C≤12)}, aryl_{(C≤12)}, heteroaryl_{(C≤12)}, heterocycloalkyl_{(C≤12)}, alkoxy_{(C≤12)}, acyloxy_{(C≤12)}, or a substituted version of any of these groups;
R₂ and R₅ are each independently hydrogen or S(O)ₓRₐ, wherein:
   x is 0, 1, or 2; and
   Rₐ is hydrogen, alkyl_{(C≤12)}, cycloalkyl_{(C≤12)}, aryl_{(C≤12)}, aralkyl_{(C≤12)}, heteroaryl_{(C≤12)}, heterocycloalkyl_{(C≤12)}, or a substituted version thereof; and
R₃ and R₄ are each independently hydrogen, alkyl_{(C≤12)}, substituted alkyl_{(C≤12)}, or —C(O)R_{b}, wherein:
   R_{b} is heterocycloalkyl_{(C≤12)}, substituted heterocycloalkyl_{(C≤12)}, heterocycloalkyl_{(C≤12)}-R_{c}, substituted heterocycloalkyl_{(C≤12)}-R_{c}; wherein:
      R_{c} is S(O)_{y}R_{c}′, wherein:
         y is 0, 1, or 2;
         R_{c}′ is hydroxy, alkyl_{(C≤12)}, cycloalkyl_{(C≤12)}, aryl_{(C≤12)}, or a substituted version of any of these groups;
            or a compound of the formula:

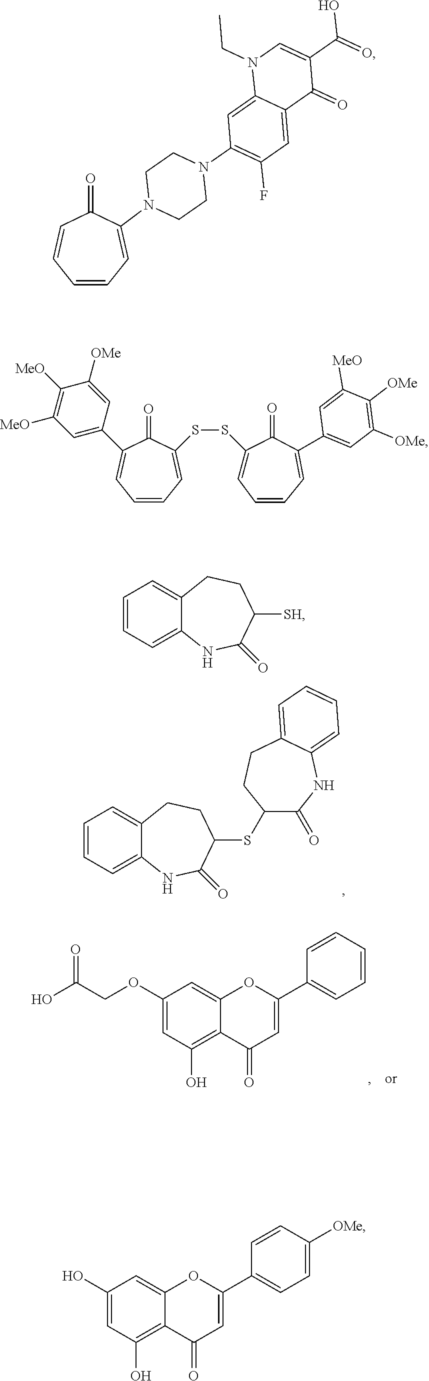

or a pharmaceutically acceptable salt thereof.

In some embodiments, the compound is further defined as:

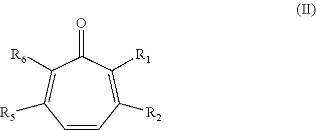

wherein:
R₁ and R₆ are each independently amino, hydroxy, mercapto, alkyl_{(C≤12)}, cycloalkyl_{(C≤12)}, aryl_{(C≤12)}, heteroaryl_{(C≤12)}, heterocycloalkyl_{(C≤12)}, alkoxy_{(C≤12)}, acyloxy_{(C≤12)}, or a substituted version of any of these groups;
R₂ and R₅ are each independently hydrogen or S(O)ₓRₐ, wherein:
   x is 0, 1, or 2; and
   Rₐ is hydrogen, alkyl_{(C≤12)}, cycloalkyl_{(C≤12)}, aryl_{(C≤12)}, aralkyl_{(C≤12)}, heteroaryl_{(C≤12)}, heterocycloalkyl_{(C≤12)}, or a substituted version thereof; and
      or a pharmaceutically acceptable salt thereof.

In some embodiments, the compound is further defined as:

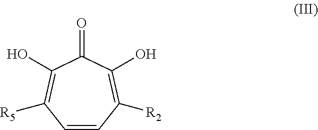

wherein:
R₂ and R₅ are each independently hydrogen or S(O)ₓRₐ, wherein:
   x is 0, 1, or 2; and
   Rₐ is hydrogen, alkyl_{(C≤12)}, cycloalkyl_{(C≤12)}, aryl_{(C≤12)}, aralkyl_{(C≤12)}, heteroaryl_{(C≤12)}, heterocycloalkyl_{(C≤12)}, or a substituted version thereof; and
      or a pharmaceutically acceptable salt thereof.

In some embodiments, R₃ is hydrogen. In other embodiments, R₃ is —C(O)R_{b}, wherein: R_{b} is heterocycloalkyl_{(C≤12)}, substituted heterocycloalkyl_{(C≤12)}, heterocycloalkyl_{(C≤12)}-R_{c}, substituted heterocycloalkyl_{(C≤12)}-R_{c}; wherein: R_{c} is S(O)_{y}R_{c}′, wherein: y is 0, 1, or 2; and R_{c}′ is hydroxy, alkyl_{(C≤12)}, cycloalkyl_{(C≤12)}, aryl_{(C≤12)}, or a substituted version of any of these groups. In some embodiments, R₄ is hydrogen. In some embodiments, R₄ is alkyl_{(C≤12)}or substituted alkyl_{(C≤12)}. In some embodiments, R₁ is hydroxy. In other embodiments, R₁ is heterocycloalkyl_{(C≤12)}or substituted heterocycloalkyl_{(C≤12)}. In other embodiments, R₁ is aryl_{(C≤12)} or substituted aryl_{(C≤12)}.

In some embodiments, R₆ is hydroxy. In other embodiments, R₆ is mercapto. In some embodiments, R₆ is amino.

In some embodiments, R₂ is S(O)ₓRₐ, wherein: x is 0, 1, or 2; and Rₐ is hydrogen, alkyl_{(C≤12)}, cycloalkyl_{(C≤12)}, aryl_{(C≤12)}, aralkyl_{(C≤12)}, heteroaryl_{(C≤12)}, heterocycloalkyl_{(C≤12)}, or a substituted version thereof. In some embodiments, x is 0. In other embodiments, x is 2. In some embodiments, Rₐ is hydrogen. In other embodiments, Rₐ is alkyl_{(C≤12)} or substituted alkyl_{(C≤12)}. In some embodiments, Rₐ is alkyl_{(C≤12)}such as butyl or hexyl. In other embodiments, Rₐ is aryl_{(C≤12)}or substituted aryl_{(C≤12)}. In some embodiments, Rₐ is aryl_{(C≤12)}such as 4-methylphenyl. In some embodiments, R₂ is hydrogen. In other embodiments, R₂ is hydrogen. In other embodiments, R₂ is S(O)ₓRₐ, wherein: x is 0, 1, or 2; and Rₐ is hydrogen, alkyl_{(C≤12)}, cycloalkyl_{(C≤12)}, aryl_{(C≤12)}, aralkyl_{(C≤12)}, heteroaryl_{(C≤12)}, heterocycloalkyl_{(C≤12)}, or a substituted version thereof. In some embodiments, x is 0. In other embodiments, x is 2. In some embodiments, Rₐ is hydrogen. In other embodiments, Rₐ is alkyl_{(C≤12)}or substituted alkyl_{(C≤12)}. In some embodiments, Rₐ is alkyl_{(C≤12)} such as butyl or hexyl. In other embodiments, Rₐ is aryl_{(C≤12)} or substituted aryl_{(C≤12)}. In some embodiments, Rₐ is aryl_{(C≤12)} such as 4-methylphenyl.

In some embodiments, the compounds are further defined as:

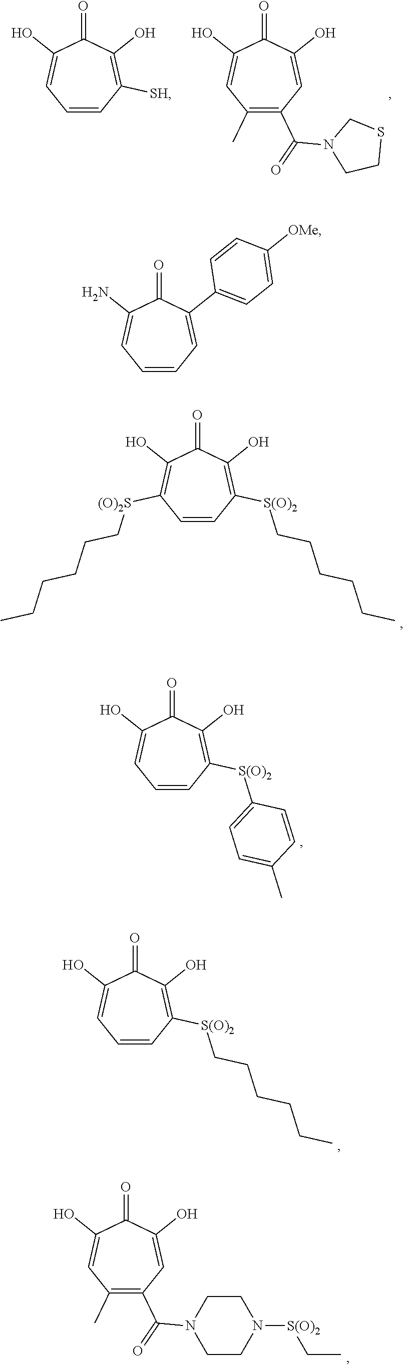

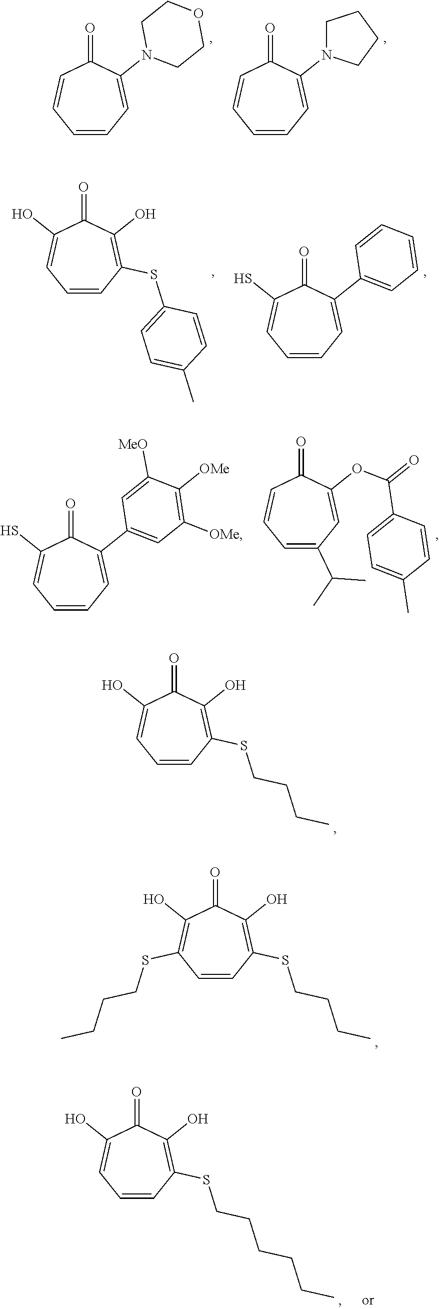

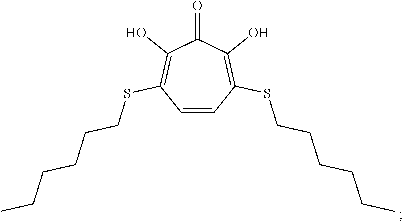 [SMILES: CCCCCCSc1ccc(SCCCCCC)c(O)c(=O)c1O]

or a pharmaceutically acceptable salt thereof.

In some embodiments, the compounds are further defined as:

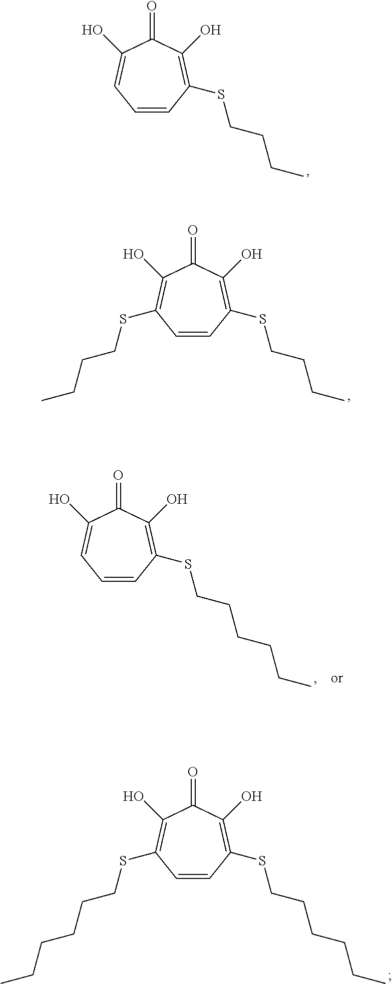

or a pharmaceutically acceptable salt thereof.

In some embodiments, the compounds are further defined as:

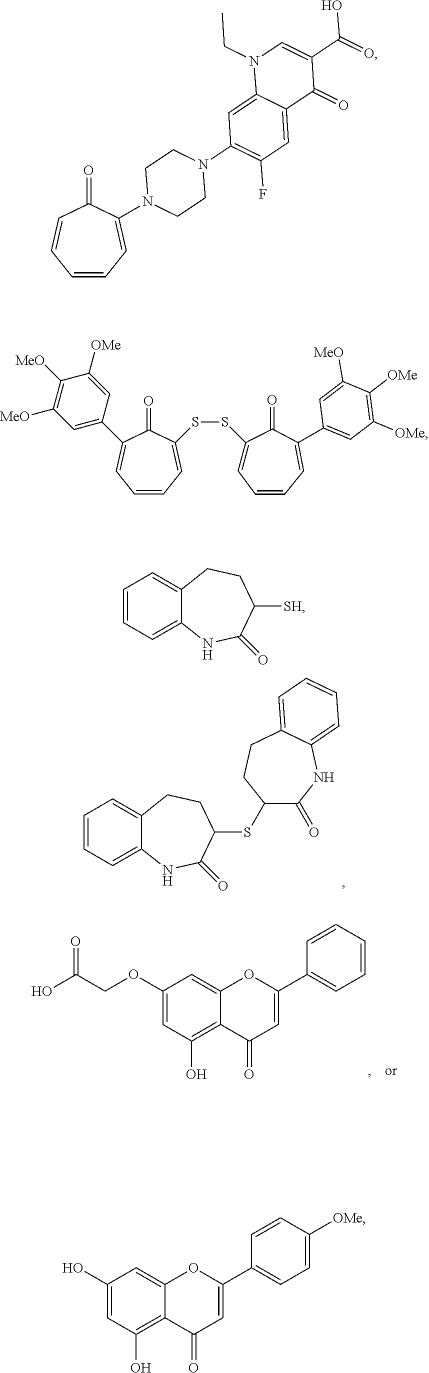

or a pharmaceutically acceptable salt thereof.

In some embodiments, the infection of a mycobacteria is an infection of Mycobacterium tuberculosis. In some embodiments, the infection of a mycobacteria is an infection of Mycobacterium avium complex. In some embodiments, the infection of Mycobacteria Tuberculosis is tuberculosis. In some embodiments, the infection of a mycobacteria is pulmonary nontuberculous mycobacteria. In some embodiments, the mycobacteria are resistant to one or more antibiotics. In some embodiments, the mycobacteria are resistant to two or more antibiotics. In some embodiments, the mycobacteria are resistant to isoniazid and rifampin. In some embodiments, the mycobacteria are resistant to at least three antibiotics. In some embodiments, the mycobacteria are resistant to either fluoroquinolones or aminoglycosides. In some embodiments, the mycobacteria are resistant to fluoroquinolones and aminoglycosides.

In some embodiments, the methods further comprise administering a second antibiotic agent. In some embodiments, the second antibiotic agent is rifampicin, rifabutin, ciprofloxacin, amikacin, ethambutol, streptomycin, clarithromycin, azithromycin, isoniazid, pyrazinamide, or rifapentine. In some embodiments, the methods comprise using two or more of rifampicin, rifabutin, ciprofloxacin, amikacin, ethambutol, streptomycin, clarithromycin, azithromycin, isoniazid, pyrazinamide, or rifapentine.

In some embodiments, the patient is a mammal such as a human. In some embodiments, the methods comprise administering the compound once. In other embodiments, the methods comprise administering the compound two or more times. In some embodiments, the methods comprise administering the compound for 1 week to 2 years. In some embodiments, the methods comprise administering the compound orally, via inhalation, or via injection. In some embodiments, the compound is administered intravenously, intramuscularly, intraperitoneally, or subcutaneously.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description. Note that simply because a particular compound is ascribed to one particular generic formula doesn't mean that it cannot also belong to another generic formula.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIGS. 1A & 1B show the structure and anti-mycobacterial activity of select troponoids. Shown are the structures (FIG. 1A) and calculated percent inhibitions of M. tuberculosis (Mtb) growth inside primary human monocytes by 4 selected troponoids (FIG. 1B). Of note, all the compounds did not have cytotoxicity on macrophages at 100 μM (the highest concentration used in the cytotoxicity assays).

FIG. 2 shows the EC₅₀ curves for compounds 694, 840, and 841.

### DETAILED DESCRIPTION OF THE PREFERRED EMBODIMENTS

The present disclosure provides compounds which are useful for the treatment of an infection of a mycobacteria such as tuberculosis and other diseases. In particular, the present disclosure provides compounds that may be used to treat a pathogenic infection such as infections caused by Mycobacterium tuberculosis (Mtb). In some embodiments, the compounds provided are used to treat tuberculosis. In some embodiments, the Mycobacterium tuberculosis (Mtb) may be a drug resistant Mycobacterium tuberculosis which is resistant to one or more of the front line antibiotic drugs such as isoniazid and rifampicin.

### I. TUBERCULOSIS

Tuberculosis (TB) is a disease caused by an infection of Mycobacterium tuberculosis (Mtb). Generally, this bacterium infects the lungs and results in a latent infection in which no discernable symptoms can be detected. In some cases, the latent condition can progress into the active form of the disease. In some estimates, infection with Mtb results in over 9 million new cases of TB and 1.5 million deaths annually (World Health Organization Global Tuberculosis Report, 2015). Some estimates have contemplated that at least a third of the world population is infected with Symptoms of an active infection include a chronic cough often associated with blood-containing sputum, fever, night sweats, and weight loss. The bacterium is transmitted through the air from patients with an active infection, while patients with a latent infection are generally not contagious.

Subjects with weakened immune system such as those with HIV/AIDS or who smoke, subjects who work in high risk environments such as hospitals, schools, or house with a person with an active infection are at high risk of contracting TB. Diagnosis occurs through the use of a latent testing protocol such as a skin test or an interferon gamma release assay but these particular tests are not useful to identifying an active infection and rather are only used to determine the presence of a latent infection. Active infections are often identified by the use of a chest X-ray or sputum cultures for acid-fast bacteria. The standard for determining the presence of an active infection though is the detection of Mtb in a clinical sample such as sputum or tissue.

Treatment of Tb involves administering to the patient a sufficient amount of a therapeutic agent such as an antibiotic. A robust antibacterial defense usually controls primary Mtb infection by reducing bacterial numbers to uncultivable levels (Medlar, 1955) but is often unable to eradicate the pathogen, resulting in a large population of latently-infected individuals that may reactivate the infection later in life. In addition to its ability to resist elimination by host immunity, Mtb infection is only slowly sterilized by antibiotic treatment. Patients that are latently infected with Mtb require 3-9 months of antibiotic therapy to prevent reactivation of infection, despite low bacterial burdens. Typical first generation treatment includes the use of a cocktail of agents. including isoniazid, rifampicin, pyrazinamide, and ethambutol. This particular cocktail is often used in four significant courses including using the four drug combination daily or at least five times a week for 8 weeks and followed by a course of isoniazid and rifampicin daily or at least five times a week for 18 weeks, using the four drug combination daily or at least five times a week for 8 weeks and followed by a course of isoniazid and rifampicin three times a week for 18 weeks, using the four drug combination three times a week for 8 weeks and followed by a course of isoniazid and rifampicin three times a week for 18 weeks, or using the four drug combination daily for 2 weeks, followed by 2 days a week for 6 weeks, and followed by a course of isoniazid and rifampicin twice a week for 18 weeks. To achieve clinical cure in greater than 90% of patients with active TB, multidrug antibiotic therapy for 6 months is required. Because of the long courses of antibiotic therapy, incomplete therapy is common and has resulted in the rise of multidrug resistant (MDR) TB cases that are resistant to at least the two frontline antibiotics used to treat TB, isoniazid (INH) and rifampicin (RIF). MDR-TB constituted 3.7% of new TB cases in 2014 and 20% of previously treated TB cases, with rates of MDR-TB as high as 48% of TB cases in some countries (World Health Organization Global Tuberculosis Report, 2015). Furthermore, extensively drug resistant TB has now been isolated in almost 80 countries throughout the world, including the US. In cases of multidrug resistant tuberculosis or other difficult to treat cases of tuberculosis, an additional agent maybe used. These agents are often divided into four different groups: Group A of fluoroquinolones including levofloxacin, moxifloxacin, or gatifloxacin, Group B of injectable anti-TB drugs including kanamycin, amikacin, streptomycin, or capreomycin, Group C of second-line agents including ethionamide, prothionamide, cycloserine, terizidone, linezolid, or clofazinime, and Group D of add-on agents including high-dose isoniazid, pyrazinamide, ethambutol, bedaquiline, delamanid, para-aminosalicylic acid, imipenem with either cilastatin or meropenem with clavulanate, or thiocetazone. Additionally, a vaccine such as the BCG vaccine may be administered in cases to prevent active infections.

### II. ACTIVE AGENTS AND INTERMEDIATES

### A. Compounds of the Present Disclosure

The compounds of the present disclosure are shown, for example, above, in the summary of the invention section, and in the claims below. They may be made using the synthetic methods outlined in the Examples section. These methods can be further modified and optimized using the principles and techniques of organic chemistry as applied by a person skilled in the art. Such principles and techniques are taught, for example, in International Patent Application No. WO 2020/227180 and Smith, March's Advanced Organic Chemistry: Reactions, Mechanisms, and Structure, (2013), which is incorporated by reference herein. In addition, the synthetic methods may be further modified and optimized for preparative, pilot- or large-scale production, either batch or continuous, using the principles and techniques of process chemistry as applied by a person skilled in the art. Such principles and techniques are taught, for example, in Anderson, Practical Process Research & Development—A Guide for Organic Chemists (2012), which is incorporated by reference herein.

All the compounds of the present invention may in some embodiments be used for the prevention and treatment of one or more diseases or disorders discussed herein or otherwise. In some embodiments, one or more of the compounds characterized or exemplified herein as an intermediate, a metabolite, and/or prodrug, may nevertheless also be useful for the prevention and treatment of one or more diseases or disorders. As such unless explicitly stated to the contrary, all the compounds of the present invention are deemed “active compounds” and “therapeutic compounds” that are contemplated for use as active pharmaceutical ingredients (APIs). Actual suitability for human or veterinary use is typically determined using a combination of clinical trial protocols and regulatory procedures, such as those administered by the Food and Drug Administration (FDA). In the United States, the FDA is responsible for protecting the public health by assuring the safety, effectiveness, quality, and security of human and veterinary drugs, vaccines and other biological products, and medical devices.

In some embodiments, the compounds of the present invention have the advantage that they may be more efficacious than, be less toxic than, be longer acting than, be more potent than, produce fewer side effects than, be more easily absorbed than, more metabolically stable than, more lipophilic than, more hydrophilic than, and/or have a better pharmacokinetic profile (e.g., higher oral bioavailability and/or lower clearance) than, and/or have other useful pharmacological, physical, or chemical properties over, compounds known in the prior art, whether for use in the indications stated herein or otherwise.

Compounds of the present invention may contain one or more asymmetrically-substituted carbon, nitrogen, sulfur, or phosphorus atom and may be isolated in optically active or racemic form. Thus, all chiral, diastereomeric, racemic form, epimeric form, and all geometric isomeric forms of a chemical formula are intended, unless the specific stereochemistry or isomeric form is specifically indicated. Compounds may occur as racemates and racemic mixtures, single enantiomers, diastereomeric mixtures and individual diastereomers. In some embodiments, a single diastereomer is obtained. The chiral centers of the compounds of the present invention can have the S or the R configuration. In some embodiments, the present compounds may contain two or more atoms which have a defined stereochemical orientation.

Chemical formulas used to represent compounds of the present invention will typically only show one of possibly several different tautomers. For example, many types of ketone groups are known to exist in equilibrium with corresponding enol groups. Similarly, many types of imine groups exist in equilibrium with enamine groups. Regardless of which tautomer is depicted for a given compound, and regardless of which one is most prevalent, all tautomers of a given chemical formula are intended.

In addition, atoms making up the compounds of the present invention are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

In some embodiments, compounds of the present invention function as prodrugs or can be derivatized to function as prodrugs. Since prodrugs are known to enhance numerous desirable qualities of pharmaceuticals (e.g., solubility, bioavailability, manufacturing, etc.), the compounds employed in some methods of the invention may, if desired, be delivered in prodrug form. Thus, the invention contemplates prodrugs of compounds of the present invention as well as methods of delivering prodrugs. Prodrugs of the compounds employed in the invention may be prepared by modifying functional groups present in the compound in such a way that the modifications are cleaved, either in routine manipulation or in vivo, to the parent compound. Accordingly, prodrugs include, for example, compounds described herein in which a hydroxy, amino, or carboxy group is bonded to any group that, when the prodrug is administered to a patient, cleaves to form a hydroxy, amino, or carboxylic acid, respectively.

In some embodiments, compounds of the present invention exist in salt or non-salt form. With regard to the salt form(s), in some embodiments the particular anion or cation forming a part of any salt form of a compound provided herein is not critical, so long as the salt, as a whole, is pharmacologically acceptable. Additional examples of pharmaceutically acceptable salts and their methods of preparation and use are presented in Handbook of Pharmaceutical Salts: Properties, and Use (2002), which is incorporated herein by reference.

It will be appreciated that many organic compounds can form complexes with solvents in which they are reacted or from which they are precipitated or crystallized. These complexes are known as “solvates.” Where the solvent is water, the complex is known as a “hydrate.” It will also be appreciated that many organic compounds can exist in more than one solid form, including crystalline and amorphous forms. All solid forms of the compounds provided herein, including any solvates thereof are within the scope of the present invention.

### B. Chemical Definitions

When used in the context of a chemical group: “hydrogen” means —H; “hydroxy” means —OH; “oxo” means ═O; “carbonyl” means —C(═O)—; “carboxy” means —C(═O)OH (also written as —COOH or —CO₂H); “halo” means independently —F, —Cl, —Br or —I; “amino” means —NH₂; “hydroxyamino” means —NHOH; “nitro” means —NO₂; imino means ═NH; “cyano” means —CN; “isocyanyl” means —N═C═O; “azido” means —N₃; in a monovalent context “phosphate” means —OP(O)(OH)₂ or a deprotonated form thereof; in a divalent context “phosphate” means —OP(O)(OH)O— or a deprotonated form thereof; “mercapto” means —SH; and “thio” means ═S; “thiocarbonyl” means —C(═S)—; “sulfonyl” means —S(O)₂—; and “sulfinyl” means —S(O)—.

In the context of chemical formulas, the symbol “—” means a single bond, “═” means a double bond, and “≡” means triple bond. The symbol “----” represents an optional bond, which if present is either single or double. The symbol “ [symbol] ” represents a single bond or a double bond. Thus, the formula

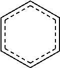

covers, for example,

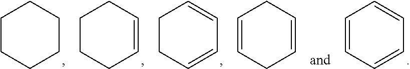

And it is understood that no one such ring atom forms part of more than one double bond. Furthermore, it is noted that the covalent bond symbol “—”, when connecting one or two stereogenic atoms, does not indicate any preferred stereochemistry. Instead, it covers all stereoisomers as well as mixtures thereof. The symbol “ [symbol] ”, when drawn perpendicularly across a bond (e.g.,

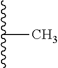

for methyl) indicates a point of attachment of the group. It is noted that the point of attachment is typically only identified in this manner for larger groups in order to assist the reader in unambiguously identifying a point of attachment. The symbol “ [symbol] ” means a single bond where the group attached to the thick end of the wedge is “out of the page.” The symbol [symbol] means a single bond where the group attached to the thick end of the wedge is “into the page”. The symbol “ [symbol] ” means a single bond where the geometry around a double bond (e.g., either E or Z) is undefined. Both options, as well as combinations thereof are therefore intended. Any undefined valency on an atom of a structure shown in this application implicitly represents a hydrogen atom bonded to that atom. A bold dot on a carbon atom indicates that the hydrogen attached to that carbon is oriented out of the plane of the paper.

When a variable is depicted as a “floating group” on a ring system, for example, the group “R” in the formula:

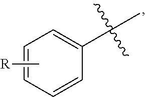

then the variable may replace any hydrogen atom attached to any of the ring atoms, including a depicted, implied, or expressly defined hydrogen, so long as a stable structure is formed. When a variable is depicted as a “floating group” on a fused ring system, as for example the group “R” in the formula:

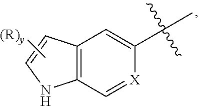

then the variable may replace any hydrogen attached to any of the ring atoms of either of the fused rings unless specified otherwise. Replaceable hydrogens include depicted hydrogens (e.g., the hydrogen attached to the nitrogen in the formula above), implied hydrogens (e.g., a hydrogen of the formula above that is not shown but understood to be present), expressly defined hydrogens, and optional hydrogens whose presence depends on the identity of a ring atom (e.g., a hydrogen attached to group X, when X equals —CH—), so long as a stable structure is formed. In the example depicted, R may reside on either the 5-membered or the 6-membered ring of the fused ring system. In the formula above, the subscript letter “y” immediately following the R enclosed in parentheses, represents a numeric variable. Unless specified otherwise, this variable can be 0, 1, 2, or any integer greater than 2, only limited by the maximum number of replaceable hydrogen atoms of the ring or ring system.

For the chemical groups and compound classes, the number of carbon atoms in the group or class is as indicated as follows: “Cn” or “C=n” defines the exact number (n) of carbon atoms in the group/class. “C≤n” defines the maximum number (n) of carbon atoms that can be in the group/class, with the minimum number as small as possible for the group/class in question. For example, it is understood that the minimum number of carbon atoms in the groups “alkyl_{(C≤8)}”, “alkanediyl_{(C≤8)}”, “heteroaryl_{(C≤8)}”, and “acyl_{(C≤8)}” is one, the minimum number of carbon atoms in the groups “alkenyl_{(C≤8)}”, “alkynyl_{(C≤8)}”, and “heterocycloalkyl_{(C≤8)}” is two, the minimum number of carbon atoms in the group “cycloalkyl_{(C≤8)}” is three, and the minimum number of carbon atoms in the groups “aryl_{(C≤8)}” and “arenediyl_{(C≤8)}” is six. “Cn-n′” defines both the minimum (n) and maximum number (n′) of carbon atoms in the group. Thus, “alkyl_{(C2-10)}” designates those alkyl groups having from 2 to 10 carbon atoms. These carbon number indicators may precede or follow the chemical groups or class it modifies and it may or may not be enclosed in parenthesis, without signifying any change in meaning. Thus, the terms “C₁₋₄-alkyl”, “C1-4-alkyl”, “alkyl_{(C1-4)}”, and “alkyl_{(C≤4)}” are all synonymous. Except as noted below, every carbon atom is counted to determine whether the group or compound falls with the specified number of carbon atoms. For example, the group dihexylamino is an example of a dialkylamino_{(C12)} group; however, it is not an example of a dialkylamino_{(C6)} group. Likewise, phenylethyl is an example of an aralkyl_{(C=8)} group. When any of the chemical groups or compound classes defined herein is modified by the term “substituted”, any carbon atom in the moiety replacing the hydrogen atom is not counted. Thus methoxyhexyl, which has a total of seven carbon atoms, is an example of a substituted alkyl_{(C1-6)}. Unless specified otherwise, any chemical group or compound class listed in a claim set without a carbon atom limit has a carbon atom limit of less than or equal to twelve.

The term “saturated” when used to modify a compound or chemical group means the compound or chemical group has no carbon-carbon double and no carbon-carbon triple bonds, except as noted below. When the term is used to modify an atom, it means that the atom is not part of any double or triple bond. In the case of substituted versions of saturated groups, one or more carbon oxygen double bond or a carbon nitrogen double bond may be present. And when such a bond is present, then carbon-carbon double bonds that may occur as part of keto-enol tautomerism or imine/enamine tautomerism are not precluded. When the term “saturated” is used to modify a solution of a substance, it means that no more of that substance can dissolve in that solution.

The term “aliphatic” signifies that the compound or chemical group so modified is an acyclic or cyclic, but non-aromatic compound or group. In aliphatic compounds/groups, the carbon atoms can be joined together in straight chains, branched chains, or non-aromatic rings (alicyclic). Aliphatic compounds/groups can be saturated, that is joined by single carbon-carbon bonds (alkanes/alkyl), or unsaturated, with one or more carbon-carbon double bonds (alkenes/alkenyl) or with one or more carbon-carbon triple bonds (alkynes/alkynyl).

The term “aromatic” signifies that the compound or chemical group so modified has a planar unsaturated ring of atoms with 4n+2 electrons in a fully conjugated cyclic π system. An aromatic compound or chemical group may be depicted as a single resonance structure; however, depiction of one resonance structure is taken to also refer to any other resonance structure. For example:

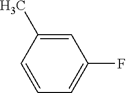

is also taken to refer to

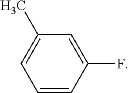

Aromatic compounds may also be depicted using a circle to represent the delocalized nature of the electrons in the fully conjugated cyclic π system, two non-limiting examples of which are shown below:

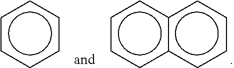

The term “alkyl” refers to a monovalent saturated aliphatic group with a carbon atom as the point of attachment, a linear or branched acyclic structure, and no atoms other than carbon and hydrogen. The groups —CH₃ (Me), —CH₂CH₃ (Et), —CH₂CH₂CH₃ (n-Pr or propyl), —CH(CH₃)₂ (i-Pr, ⁱPr or isopropyl), —CH₂CH₂CH₂CH₃ (n-Bu), —CH(CH₃)CH₂CH₃ (sec-butyl), —CH₂CH(CH₃)₂ (isobutyl), —C(CH₃)₃ (tert-butyl, t-butyl, t-Bu or ^{t}Bu), and —CH₂C(CH₃)₃ (neo-pentyl) are non-limiting examples of alkyl groups. The term “alkanediyl” refers to a divalent saturated aliphatic group, with one or two saturated carbon atom(s) as the point(s) of attachment, a linear or branched acyclic structure, no carbon-carbon double or triple bonds, and no atoms other than carbon and hydrogen. The groups —CH₂— (methylene), —CH₂CH_{b 2}—, —CH₂C(CH₃)₂CH₂—, and —CH₂CH₂CH₂— are non-limiting examples of alkanediyl groups. The term “alkylidene” refers to the divalent group ═CRR′ in which R and R′ are independently hydrogen or alkyl. Non-limiting examples of alkylidene groups include: ═CH₂, ═CH(CH₂CH₃), and ═C(CH₃)₂. An “alkane” refers to the class of compounds having the formula H—R, wherein R is alkyl as this term is defined above.

The term “cycloalkyl” refers to a monovalent saturated aliphatic group with a carbon atom as the point of attachment, said carbon atom forming part of one or more non-aromatic ring structures, no carbon-carbon double or triple bonds, and no atoms other than carbon and hydrogen. If more than one ring is present, the rings may be fused, bridged, or spirocyclic. Non-limiting examples include: —CH(CH₂)₂ (cyclopropyl), cyclobutyl, cyclopentyl, or cyclohexyl (Cy). As used herein, the term does not preclude the presence of one or more alkyl groups (carbon number limitation permitting) attached to a carbon atom of the non-aromatic ring structure. The term “cycloalkanediyl” refers to a divalent saturated aliphatic group with two carbon atoms as points of attachment, no carbon-carbon double or triple bonds, and no atoms other than carbon and hydrogen. The group

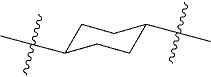 [SMILES: *C1CCC(*)CC1]

is a non-limiting example of cycloalkanediyl group. A “cycloalkane” refers to the class of compounds having the formula H—R, wherein R is cycloalkyl as this term is defined above.

The term “alkenyl” refers to a monovalent unsaturated aliphatic group with a carbon atom as the point of attachment, a linear or branched, acyclic structure, at least one nonaromatic carbon-carbon double bond, no carbon-carbon triple bonds, and no atoms other than carbon and hydrogen. Non-limiting examples include: —CH═CH₂ (vinyl), —CH═CHCH₃, —CH═CHCH₂CH₃, —CH₂CH═CH₂ (allyl), —CH₂CH═CHCH₃, and —CH═CHCH═CH₂. The term “alkenediyl” refers to a divalent unsaturated aliphatic group, with two carbon atoms as points of attachment, a linear or branched acyclic structure, at least one nonaromatic carbon-carbon double bond, no carbon-carbon triple bonds, and no atoms other than carbon and hydrogen. The groups —CH═CH—, —CH═C(CH₃)CH₂—, —CH═CHCH₂—, and —CH₂CH═CHCH₂— are non-limiting examples of alkenediyl groups. It is noted that while the alkenediyl group is aliphatic, once connected at both ends, this group is not precluded from forming part of an aromatic structure. The terms “alkene” and “olefin” are synonymous and refer to the class of compounds having the formula H—R, wherein R is alkenyl as this term is defined above. Similarly, the terms “terminal alkene” and “α-olefin” are synonymous and refer to an alkene having just one carbon-carbon double bond, wherein that bond is part of a vinyl group at an end of the molecule.

The term “alkynyl” refers to a monovalent unsaturated aliphatic group with a carbon atom as the point of attachment, a linear or branched acyclic structure, at least one carbon-carbon triple bond, and no atoms other than carbon and hydrogen. As used herein, the term alkynyl does not preclude the presence of one or more non-aromatic carbon-carbon double bonds. The groups —C≡CH, —C≡CCH₃, and —CH₂C≡CCH₃ are non-limiting examples of alkynyl groups. An “alkyne” refers to the class of compounds having the formula H—R, wherein R is alkynyl.

The term “aryl” refers to a monovalent unsaturated aromatic group with an aromatic carbon atom as the point of attachment, said carbon atom forming part of a one or more aromatic ring structures, each with six ring atoms that are all carbon, and wherein the group consists of no atoms other than carbon and hydrogen. If more than one ring is present, the rings may be fused or unfused. Unfused rings are connected with a covalent bond. As used herein, the term aryl does not preclude the presence of one or more alkyl groups (carbon number limitation permitting) attached to the first aromatic ring or any additional aromatic ring present. Non-limiting examples of aryl groups include phenyl (Ph), methylphenyl, (dimethyl)phenyl, —C₆H₄CH₂CH₃ (ethylphenyl), naphthyl, and a monovalent group derived from biphenyl (e.g., 4-phenylphenyl). The term “arenediyl” refers to a divalent aromatic group with two aromatic carbon atoms as points of attachment, said carbon atoms forming part of one or more six-membered aromatic ring structures, each with six ring atoms that are all carbon, and wherein the divalent group consists of no atoms other than carbon and hydrogen. As used herein, the term arenediyl does not preclude the presence of one or more alkyl groups (carbon number limitation permitting) attached to the first aromatic ring or any additional aromatic ring present. If more than one ring is present, the rings may be fused or unfused. Unfused rings are connected with a covalent bond. Non-limiting examples of arenediyl groups include:

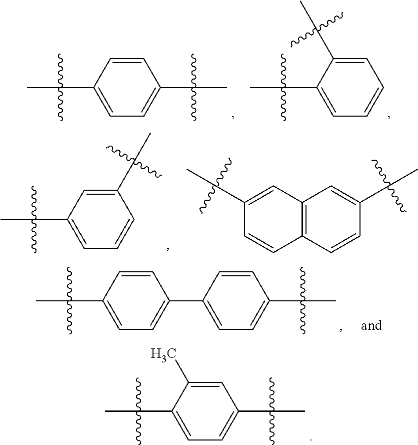

An “arene” refers to the class of compounds having the formula H—R, wherein R is aryl as that term is defined above. Benzene and toluene are non-limiting examples of arenes.

The term “aralkyl” refers to the monovalent group -alkanediyl- aryl, in which the terms alkanediyl and aryl are each used in a manner consistent with the definitions provided above. Non-limiting examples are: phenylmethyl (benzyl, Bn) and 2-phenyl-ethyl.

The term “heteroaryl” refers to a monovalent aromatic group with an aromatic carbon atom or nitrogen atom as the point of attachment, said carbon atom or nitrogen atom forming part of one or more aromatic ring structures, each with three to eight ring atoms, wherein at least one of the ring atoms of the aromatic ring structure(s) is nitrogen, oxygen or sulfur, and wherein the heteroaryl group consists of no atoms other than carbon, hydrogen, aromatic nitrogen, aromatic oxygen and aromatic sulfur. If more than one ring is present, the rings are fused; however, the term heteroaryl does not preclude the presence of one or more alkyl or aryl groups (carbon number limitation permitting) attached to one or more ring atoms. Non-limiting examples of heteroaryl groups include benzoxazolyl, benzimidazolyl, furanyl, imidazolyl (Im), indolyl, indazolyl, isoxazolyl, methylpyridinyl, oxazolyl, oxadiazolyl, phenylpyridinyl, pyridinyl (pyridyl), pyrrolyl, pyrimidinyl, pyrazinyl, quinolyl, quinazolyl, quinoxalinyl, triazinyl, tetrazolyl, thiazolyl, thienyl, and triazolyl. The term “N-heteroaryl” refers to a heteroaryl group with a nitrogen atom as the point of attachment. A “heteroarene” refers to the class of compounds having the formula H—R, wherein R is heteroaryl. Pyridine and quinoline are non-limiting examples of heteroarenes.

The term “heterocycloalkyl” refers to a monovalent non-aromatic group with a carbon atom or nitrogen atom as the point of attachment, said carbon atom or nitrogen atom forming part of one or more non-aromatic ring structures, each with three to eight ring atoms, wherein at least one of the ring atoms of the non-aromatic ring structure(s) is nitrogen, oxygen or sulfur, and wherein the heterocycloalkyl group consists of no atoms other than carbon, hydrogen, nitrogen, oxygen and sulfur. If more than one ring is present, the rings may be fused, bridged, or spirocyclic. As used herein, the term does not preclude the presence of one or more alkyl groups (carbon number limitation permitting) attached to one or more ring atoms. Also, the term does not preclude the presence of one or more double bonds in the ring or ring system, provided that the resulting group remains non-aromatic. Non-limiting examples of heterocycloalkyl groups include aziridinyl, azetidinyl, pyrrolidinyl, piperidinyl, piperazinyl, morpholinyl, thiomorpholinyl, tetrahydrofuranyl, tetrahydrothiofuranyl, tetrahydropyranyl, tetrahydropyridinyl, pyranyl, oxiranyl, and oxetanyl. The term “N-heterocycloalkyl” refers to a heterocycloalkyl group with a nitrogen atom as the point of attachment. N-pyrrolidinyl is an example of such a group.

The term “acyl” refers to the group —C(O)R, in which R is a hydrogen, alkyl, cycloalkyl, or aryl as those terms are defined above. The groups, —CHO, —C(O)CH₃ (acetyl, Ac), —C(O)CH₂CH₃, —C(O)CH(CH₃)₂, —C(O)CH(CH₂)₂, —C(O)C₆H₅, and —C(O)C₆H₄CH₃ are non-limiting examples of acyl groups. A “thioacyl” is defined in an analogous manner, except that the oxygen atom of the group —C(O)R has been replaced with a sulfur atom, —C(S)R. The term “aldehyde” corresponds to an alkyl group, as defined above, attached to a —CHO group.

The term “alkoxy” refers to the group —OR, in which R is an alkyl, as that term is defined above. Non-limiting examples include: —OCH₃ (methoxy), —OCH₂CH₃ (ethoxy), —OCH₂CH₂CH₃, —OCH(CH₃)₂ (isopropoxy), or —OC(CH₃)₃ (tert-butoxy). The terms “cycloalkoxy”, “alkenyloxy”, “alkynyloxy”, “aryloxy”, “aralkoxy”, “heteroaryloxy”, “heterocycloalkoxy”, and “acyloxy”, when used without the “substituted” modifier, refers to groups, defined as —OR, in which R is cycloalkyl, alkenyl, alkynyl, aryl, aralkyl, heteroaryl, heterocycloalkyl, and acyl, respectively. The term “alkylthio” and “acylthio” refers to the group —SR, in which R is an alkyl and acyl, respectively. The term “alcohol” corresponds to an alkane, as defined above, wherein at least one of the hydrogen atoms has been replaced with a hydroxy group. The term “ether” corresponds to an alkane, as defined above, wherein at least one of the hydrogen atoms has been replaced with an alkoxy group.

The term “alkylamino” refers to the group —NHR, in which R is an alkyl, as that term is defined above. Non-limiting examples include: —NHCH₃ and —NHCH₂CH₃. The term “dialkylamino” refers to the group —NRR′, in which R and R′ can be the same or different alkyl groups. Non-limiting examples of dialkylamino groups include: —N(CH₃)₂ and —N(CH₃)(CH₂CH₃). The term “amido” (acylamino), when used without the “substituted” modifier, refers to the group —NHR, in which R is acyl, as that term is defined above. A non-limiting example of an amido group is —NHC(O)CH₃.

When a chemical group is used with the “substituted” modifier, one or more hydrogen atom has been replaced, independently at each instance, by —OH, —F, —Cl, —Br, —I, —NH₂, —NO₂, —CO₂H, —CO₂CH₃, —CO₂CH₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. For example, the following groups are non-limiting examples of substituted alkyl groups: —CH₂OH, —CH₂Cl, —CF₃, —CH₂CN, —CH₂C(O)OH, —CH₂C(O)OCH₃, —CH₂C(O)NH₂, —CH₂C(OCH₃, —CH₂OCH₃, —CH₂OC(O)CH₃, —CH₂NH₂, —CH₂N(CH₃)₂, and —CH₂CH₂Cl. The term “haloalkyl” is a subset of substituted alkyl, in which the hydrogen atom replacement is limited to halo (i.e. —F, —Cl, —Br, or —I) such that no other atoms aside from carbon, hydrogen and halogen are present. The group, —CH₂Cl is a non-limiting example of a haloalkyl. The term “fluoroalkyl” is a subset of substituted alkyl, in which the hydrogen atom replacement is limited to fluoro such that no other atoms aside from carbon, hydrogen and fluorine are present. The groups —CH₂F, —CF₃, and —CH₂CF₃ are non-limiting examples of fluoroalkyl groups. Non-limiting examples of substituted aralkyls are: (3-chlorophenyl)-methyl, and 2-chloro-2-phenyl-eth-1-yl. The groups, —C(O)CH₂CF₃, —CO₂H (carboxyl), —CO₂CH₃ (methylcarboxyl), —CO₂CH₂CH₃, —C(O)NH₂ (carbamoyl), and —CON(CH₃)₂, are non-limiting examples of substituted acyl groups. The groups —NHC(O)OCH₃ and —NHC(O)NHCH₃ are non-limiting examples of substituted amido groups.

The use of the word “a” or “an,” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the device, the method being employed to determine the value, or the variation that exists among the study subjects or patients. When used in other contexts, the term “about” is used to indicate a value of ±10% of the reported value, preferably a value of ±5% of the reported value. It is to be understood that, whenever the term “about” is used, a specific reference to the exact numerical value indicated is also included.

An “active ingredient” (AI) or active pharmaceutical ingredient (API) (also referred to as an active compound, active substance, active agent, pharmaceutical agent, agent, biologically active molecule, or a therapeutic compound) is the ingredient in a pharmaceutical drug that is biologically active.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “effective,” as that term is used in the specification and/or claims, means adequate to accomplish a desired, expected, or intended result. “Effective amount,” “Therapeutically effective amount” or “pharmaceutically effective amount” when used in the context of treating a patient or subject with a compound means that amount of the compound which, when administered to the patient or subject, is sufficient to effect such treatment or prevention of the disease as those terms are defined below.

An “excipient” is a pharmaceutically acceptable substance formulated along with the active ingredient(s) of a medication, pharmaceutical composition, formulation, or drug delivery system. Excipients may be used, for example, to stabilize the composition, to bulk up the composition (thus often referred to as “bulking agents,” “fillers,” or “diluents” when used for this purpose), or to confer a therapeutic enhancement on the active ingredient in the final dosage form, such as facilitating drug absorption, reducing viscosity, or enhancing solubility. Excipients include pharmaceutically acceptable versions of antiadherents, binders, coatings, colors, disintegrants, flavors, glidants, lubricants, preservatives, sorbents, sweeteners, and vehicles. The main excipient that serves as a medium for conveying the active ingredient is usually called the vehicle. Excipients may also be used in the manufacturing process, for example, to aid in the handling of the active substance, such as by facilitating powder flowability or non-stick properties, in addition to aiding in vitro stability such as prevention of denaturation or aggregation over the expected shelf life. The suitability of an excipient will typically vary depending on the route of administration, the dosage form, the active ingredient, as well as other factors.

The term “hydrate” when used as a modifier to a compound means that the compound has less than one (e.g., hemihydrate), one (e.g., monohydrate), or more than one (e.g., dihydrate) water molecules associated with each compound molecule, such as in solid forms of the compound.

As used herein, the term “IC₅₀” refers to an inhibitory dose which is 50% of the maximum response obtained. This quantitative measure indicates how much of a particular drug or other substance (inhibitor) is needed to inhibit a given biological, biochemical or chemical process (or component of a process, i.e. an enzyme, cell, cell receptor or microorganism) by half.

An “isomer” of a first compound is a separate compound in which each molecule contains the same constituent atoms as the first compound, but where the configuration of those atoms in three dimensions differs.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate. Non-limiting examples of human patients are adults, juveniles, infants and fetuses.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds disclosed herein which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3 -phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

A “pharmaceutically acceptable carrier,” “drug carrier,” or simply “carrier” is a pharmaceutically acceptable substance formulated along with the active ingredient medication that is involved in carrying, delivering and/or transporting a chemical agent. Drug carriers may be used to improve the delivery and the effectiveness of drugs, including for example, controlled-release technology to modulate drug bioavailability, decrease drug metabolism, and/or reduce drug toxicity. Some drug carriers may increase the effectiveness of drug delivery to the specific target sites. Examples of carriers include: liposomes, microspheres (e.g., made of poly(lactic-co-glycolic) acid), albumin microspheres, synthetic polymers, nanofibers, protein-DNA complexes, protein conjugates, erythrocytes, virosomes, and dendrimers.

A “pharmaceutical drug” (also referred to as a pharmaceutical, pharmaceutical preparation, pharmaceutical composition, pharmaceutical formulation, pharmaceutical product, medicinal product, medicine, medication, medicament, or simply a drug, agent, or preparation) is a composition used to diagnose, cure, treat, or prevent disease, which comprises an active pharmaceutical ingredient (API) (defined above) and optionally contains one or more inactive ingredients, which are also referred to as excipients (defined above).

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

“Prodrug” means a compound that is convertible in vivo metabolically into an active pharmaceutical ingredient of the present invention. The prodrug itself may or may not have activity in its prodrug form. For example, a compound comprising a hydroxy group may be administered as an ester that is converted by hydrolysis in vivo to the hydroxy compound. Non-limiting examples of suitable esters that may be converted in vivo into hydroxy compounds include acetates, citrates, lactates, phosphates, tartrates, malonates, oxalates, salicylates, propionates, succinates, fumarates, maleates, methylene-bis-β-hydroxynaphthoate, gentisates, isethionates, di-p-toluoyltartrates, methanesulfonates, ethanesulfonates, benzenesulfonates, p-toluenesulfonates, cyclohexylsulfamates, quinates, and esters of amino acids. Similarly, a compound comprising an amine group may be administered as an amide that is converted by hydrolysis in vivo to the amine compound.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers. Chiral molecules contain a chiral center, also referred to as a stereocenter or stereogenic center, which is any point, though not necessarily an atom, in a molecule bearing groups such that an interchanging of any two groups leads to a stereoisomer. In organic compounds, the chiral center is typically a carbon, phosphorus or sulfur atom, though it is also possible for other atoms to be stereocenters in organic and inorganic compounds. A molecule can have multiple stereocenters, giving it many stereoisomers. In compounds whose stereoisomerism is due to tetrahedral stereogenic centers (e.g., tetrahedral carbon), the total number of hypothetically possible stereoisomers will not exceed 2ⁿ, where n is the number of tetrahedral stereocenters. Molecules with symmetry frequently have fewer than the maximum possible number of stereoisomers. A 50:50 mixture of enantiomers is referred to as a racemic mixture. Alternatively, a mixture of enantiomers can be enantiomerically enriched so that one enantiomer is present in an amount greater than 50%. Typically, enantiomers and/or diastereomers can be resolved or separated using techniques known in the art. It is contemplated that that for any stereocenter or axis of chirality for which stereochemistry has not been defined, that stereocenter or axis of chirality can be present in its R form, S form, or as a mixture of the R and S forms, including racemic and non-racemic mixtures. As used herein, the phrase “substantially free from other stereoisomers” means that the composition contains ≤15%, more preferably ≤10%, even more preferably ≤5%, or most preferably ≤1% of another stereoisomer(s).

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease or symptom thereof in a subject or patient that is experiencing or displaying the pathology or symptomatology of the disease.

The term “unit dose” refers to a formulation of the compound or composition such that the formulation is prepared in a manner sufficient to provide a single therapeutically effective dose of the active ingredient to a patient in a single administration. Such unit dose formulations that may be used include but are not limited to a single tablet, capsule, or other oral formulations, or a single vial with a syringeable liquid or other injectable formulations.

The above definitions supersede any conflicting definition in any reference that is incorporated by reference herein. The fact that certain terms are defined, however, should not be considered as indicative that any term that is undefined is indefinite. Rather, all terms used are believed to describe the disclosure in terms such that one of ordinary skill can appreciate the scope and practice the present disclosure.

### III. THERAPEUTIC METHODS

### A. Pharmaceutical Formulations and Routes of Administration

In another aspect, for administration to a patient in need of such treatment, pharmaceutical formulations (also referred to as a pharmaceutical preparations, pharmaceutical compositions, pharmaceutical products, medicinal products, medicines, medications, or medicaments) comprise a therapeutically effective amount of a compound disclosed herein formulated with one or more excipients and/or drug carriers appropriate to the indicated route of administration. In some embodiments, the compounds disclosed herein are formulated in a manner amenable for the treatment of human and/or veterinary patients. In some embodiments, formulation comprises admixing or combining one or more of the compounds disclosed herein with one or more of the following excipients: lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia, sodium alginate, polyvinylpyrrolidone, and/or polyvinyl alcohol. In some embodiments, e.g., for oral administration, the pharmaceutical formulation may be tableted or encapsulated. In some embodiments, the compounds may be dissolved or slurried in water, polyethylene glycol, propylene glycol, ethanol, corn oil, cottonseed oil, peanut oil, sesame oil, benzyl alcohol, sodium chloride, and/or various buffers. In some embodiments, the pharmaceutical formulations may be subjected to pharmaceutical operations, such as sterilization, and/or may contain drug carriers and/or excipients such as preservatives, stabilizers, wetting agents, emulsifiers, encapsulating agents such as lipids, dendrimers, polymers, proteins such as albumin, nucleic acids, and buffers.

Pharmaceutical formulations may be administered by a variety of methods, e.g., orally or by injection (e.g. subcutaneous, intravenous, and intraperitoneal). Depending on the route of administration, the compounds disclosed herein may be coated in a material to protect the compound from the action of acids and other natural conditions which may inactivate the compound. To administer the active compound by other than parenteral administration, it may be necessary to coat the compound with, or co-administer the compound with, a material to prevent its inactivation. In some embodiments, the active compound may be administered to a patient in an appropriate carrier, for example, liposomes, or a diluent. Pharmaceutically acceptable diluents include saline and aqueous buffer solutions. Liposomes include water-in-oil-in-water CGF emulsions as well as conventional liposomes.

The compounds disclosed herein may also be administered parenterally, intraperitoneally, intraspinally, or intracerebrally. Dispersions can be prepared in glycerol, liquid polyethylene glycols, and mixtures thereof and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersion. The carrier can be a solvent or dispersion medium containing, for example, water, ethanol, polyol (such as, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions can be brought about by including in the composition an agent which delays absorption, for example, aluminum monostearate or gelatin.

The compounds disclosed herein can be administered orally, for example, with an inert diluent or an assimilable edible carrier. The compounds and other ingredients may also be enclosed in a hard or soft-shell gelatin capsule, compressed into tablets, or incorporated directly into the patient's diet. For oral therapeutic administration, the compounds disclosed herein may be incorporated with excipients and used in the form of ingestible tablets, buccal tablets, troches, capsules, elixirs, suspensions, syrups, wafers, and the like. The percentage of the therapeutic compound in the compositions and preparations may, of course, be varied. The amount of the therapeutic compound in such pharmaceutical formulations is such that a suitable dosage will be obtained.

The therapeutic compound may also be administered topically to the skin, eye, ear, or mucosal membranes. Administration of the therapeutic compound topically may include formulations of the compounds as a topical solution, lotion, cream, ointment, gel, foam, transdermal patch, or tincture. When the therapeutic compound is formulated for topical administration, the compound may be combined with one or more agents that increase the permeability of the compound through the tissue to which it is administered. In other embodiments, it is contemplated that the topical administration is administered to the eye. Such administration may be applied to the surface of the cornea, conjunctiva, or sclera. Without wishing to be bound by any theory, it is believed that administration to the surface of the eye allows the therapeutic compound to reach the posterior portion of the eye. Ophthalmic topical administration can be formulated as a solution, suspension, ointment, gel, or emulsion. Finally, topical administration may also include administration to the mucosa membranes such as the inside of the mouth. Such administration can be directly to a particular location within the mucosal membrane such as a tooth, a sore, or an ulcer. Alternatively, if local delivery to the lungs is desired the therapeutic compound may be administered by inhalation in a dry-powder or aerosol formulation.

In some embodiments, it may be advantageous to formulate parenteral compositions in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical carrier. In some embodiments, the specification for the dosage unit forms of the invention are dictated by and directly dependent on (a) the unique characteristics of the therapeutic compound and the particular therapeutic effect to be achieved, and (b) the limitations inherent in the art of compounding such a therapeutic compound for the treatment of a selected condition in a patient. In some embodiments, active compounds are administered at a therapeutically effective dosage sufficient to treat a condition associated with a condition in a patient. For example, the efficacy of a compound can be evaluated in an animal model system that may be predictive of efficacy in treating the disease in a human or another animal.

In some embodiments, the effective dose range for the therapeutic compound can be extrapolated from effective doses determined in animal studies for a variety of different animals. In some embodiments, the human equivalent dose (HED) in mg/kg can be calculated in accordance with the following formula (see, e.g., Reagan-Shaw et al., FASEB J., 22(3):659-661, 2008, which is incorporated herein by reference):

HED(mg/kg)=Animal dose(mg/kg)×(Animal Kₘ/Human Kₘ)

Use of the Kₘ factors in conversion results in HED values based on body surface area (BSA) rather than only on body mass. Kₘ values for humans and various animals are well known. For example, the Kₘ for an average 60 kg human (with a BSA of 1.6 m²) is 37, whereas a 20 kg child (BSA 0.8 m²) would have a Kₘ of 25. Kₘ for some relevant animal models are also well known, including: mice Kₘ of 3 (given a weight of 0.02 kg and BSA of 0.007); hamster Kₘ of 5 (given a weight of 0.08 kg and BSA of 0.02); rat Kₘ of 6 (given a weight of 0.15 kg and BSA of 0.025) and monkey Kₘ of 12 (given a weight of 3 kg and BSA of 0.24).

Precise amounts of the therapeutic composition depend on the judgment of the practitioner and are specific to each individual. Nonetheless, a calculated HED dose provides a general guide. Other factors affecting the dose include the physical and clinical state of the patient, the route of administration, the intended goal of treatment and the potency, stability and toxicity of the particular therapeutic formulation.

The actual dosage amount of a compound of the present disclosure or composition comprising a compound of the present disclosure administered to a patient may be determined by physical and physiological factors such as type of animal treated, age, sex, body weight, severity of condition, the type of disease being treated, previous or concurrent therapeutic interventions, idiopathy of the patient and on the route of administration. These factors may be determined by a skilled artisan. The practitioner responsible for administration will typically determine the concentration of active ingredient(s) in a composition and appropriate dose(s) for the individual patient. The dosage may be adjusted by the individual physician in the event of any complication.

In some embodiments, the therapeutically effective amount typically will vary from about 0.001 mg/kg to about 1000 mg/kg, from about 0.01 mg/kg to about 750 mg/kg, from about 100 mg/kg to about 500 mg/kg, from about 1 mg/kg to about 250 mg/kg, from about 10 mg/kg to about 150 mg/kg in one or more dose administrations daily, for one or several days (depending of course of the mode of administration and the factors discussed above). Other suitable dose ranges include 1 mg to 10,000 mg per day, 100 mg to 10,000 mg per day, 500 mg to 10,000 mg per day, and 500 mg to 1,000 mg per day. In some embodiments, the amount is less than 10,000 mg per day with a range of 750 mg to 9,000 mg per day.

In some embodiments, the amount of the active compound in the pharmaceutical formulation is from about 2 to about 75 weight percent. In some of these embodiments, the amount if from about 25 to about 60 weight percent.

Single or multiple doses of the agents are contemplated. Desired time intervals for delivery of multiple doses can be determined by one of ordinary skill in the art employing no more than routine experimentation. As an example, patients may be administered two doses daily at approximately 12-hour intervals. In some embodiments, the agent is administered once a day.

The agent(s) may be administered on a routine schedule. As used herein a routine schedule refers to a predetermined designated period of time. The routine schedule may encompass periods of time which are identical, or which differ in length, as long as the schedule is predetermined. For instance, the routine schedule may involve administration twice a day, every day, every two days, every three days, every four days, every five days, every six days, a weekly basis, a monthly basis or any set number of days or weeks there-between. Alternatively, the predetermined routine schedule may involve administration on a twice daily basis for the first week, followed by a daily basis for several months, etc. In other embodiments, the invention provides that the agent(s) may be taken orally and that the timing of which is or is not dependent upon food intake. Thus, for example, the agent can be taken every morning and/or every evening, regardless of when the patient has eaten or will eat.

### B. Combination Therapy

In addition to being used as a monotherapy, the compounds of the present disclosure may also find use in combination therapies. Effective combination therapy may be achieved with a single composition or pharmacological formulation that includes both agents, or with two distinct compositions or formulations, administered at the same time, wherein one composition includes a compound of this disclosure, and the other includes the second agent(s). Alternatively, the therapy may precede or follow the other agent treatment by intervals ranging from minutes to months.

It is contemplated that any antibiotic may be administered in combination with the compounds of the present disclosure in order to treat a TB infection. In some cases, the TB infection may be a drug resistant strain which may be treated with a combination of multiple antibiotics. Some exemplary antibiotics include isoniazid, pyrazinamide, rifampicin, ethambutol, levofloxacin, moxifloxacin, gatifloxacin, kanamycin, amikacin, capreomycin, streptomycin, ethionamide, prothionamide, cycloserine, terizidone, linezolid, clofazimine, bedaquiline, delamanid, para-aminosalicylic acid, imipenem, cilastatin, meropenem, or thiocetazone. In particular embodiments, the combination methods may comprise treating with one or more of rifampicin, pyrazinamide, ethambutol, and isoniazid. In some embodiments, a therapy may comprise all four of these antibiotics. Additionally, if resistance to one of these two antibiotics is detected, then bedaquiline or linezolid may also be administered instead of one or the above noted antibiotics.

Finally, given the difficulty in treating TB infections, such combination therapies may be used for multiple months. Extremely resistant TB infections may be treated for 1 to 3 years in order to completely rid the body of the Mycobacterium tuberculosis bacterium completely. For less extensive or less difficult bacterial strains to treat, the treatments may also from 3 to 12 months instead of 1 to 3 years.

### IV. EXAMPLES

All of the compositions and/or methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and/or methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### Example 1

### Effect of Compounds on Mycobacterial Growth

### A. General Methods

Previous work has established methods to synthesize troponoids (Elagawany et al., 2018), test cytotoxicity (Abate et al., 2015; Kumar et al., 2016; Kumar et al., 2018), measure in vitro anti-mycobacterial activities (Abate et al., 2015; Kumar et al., 2016; Kumar et al., 2018), predict absorption, distribution, metabolism and excretion (ADME) (Elagawany et al., 2018), test metabolism in vitro using microsomal enzymes (Meyers et al., 2014), study pharmacokinetics (PK) in animals (Meyers et al., 2014) and determine efficacy against mycobacterial infection in mice (Meyers et al., 2014; Eickhoff et al., 2019; Blazevic et al., 2014).

### B. Initial Analysis

The initial results indicate that the troponoids described herein have excellent antimycobacterial activities. Troponoids are unique compounds which have no structural similarities to any of the anti-mycobacterial drugs used in clinical practice. The antibacterial mechanism of action of troponoids is not known but there are findings from studies on other bacteria which suggest that troponoids are iron chelators (Akers et al., 1980) and interfere with the integrity of the bacterial cell wall/cell membrane, leading to loss of cell contents (Zhao 2007). Therefore, existing resistance to known anti-TB or anti-MAC drugs are unlikely to have any effect on the activity of troponoids.

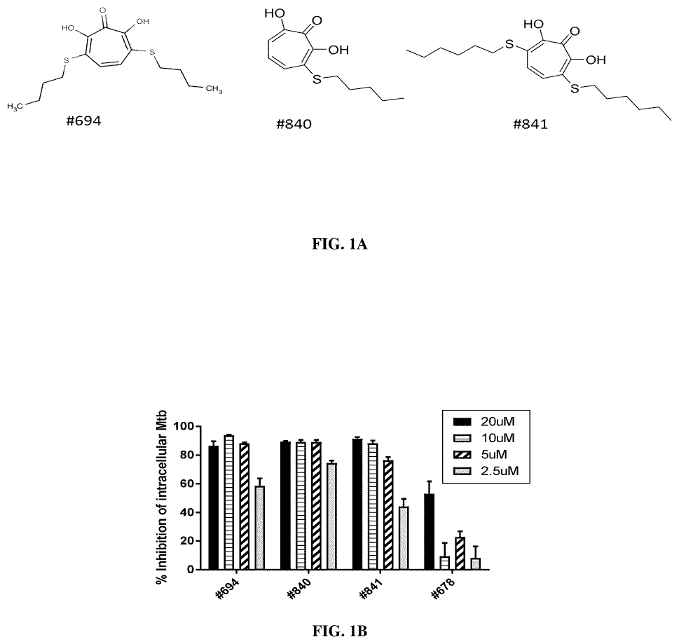

Numerous troponoids have been synthesized and screened on Mtb and MAC and identified three with activities at a concentration of ≤5 μM. FIG. 1 shows the structures of selected troponoids and their activities on intracellular mycobacteria. Briefly, primary monocytes were infected with mycobacteria (Mtb) for 4 hours, then co-cultured with indicated troponoids (2.5-20 μM) for 3 days. Then mammalian cells were lysed with saponin and residual mycobacteria quantified via ³H-Uridine as previously described (Abate et al. 2015). The potent activities on intracellular mycobacteria indicate the potential to be highly active in vivo. After these initial hits, other troponoid compounds were analyzed and are shown in Table 1 below.

TABLE 1
Additional Troponoid Compounds

| | | Activity on |
|---|---|---|
| Compound | | intracellular BCG |
| Name | Compound Structure | (% inhibition)¹ |
| #693 | 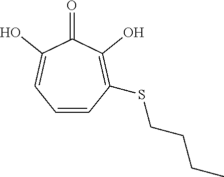 | 89.2 (not tested on intracellular Mtb) |
| #694 | 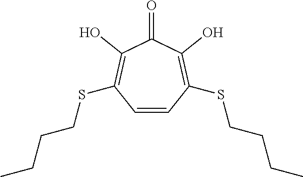 [SMILES: CCCCSc1ccc(SCCCC)c(O)c(=O)c1O] | 91.8 (86.5% on intracellular Mtb) |
| #697 | 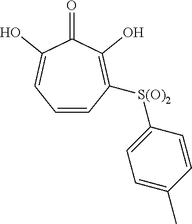 | 0 |
| #699 | 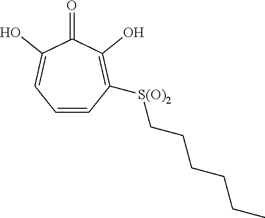 | 0 |
| #700 | 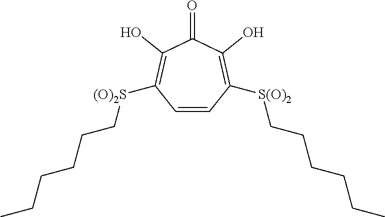 | 6.1 |
| #701 | 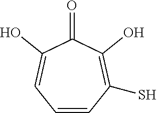 [SMILES: O=c1c(O)cccc(S)c1O] | 38.8 |
| #712 | 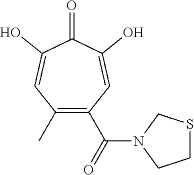 [SMILES: Cc1cc(O)c(=O)c(O)cc1C(=O)N1CCSC1] | 32.1 |
| #762 | 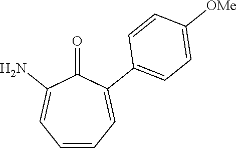 [SMILES: COc1ccc(-c2ccccc(N)c2=O)cc1] | 10.8 |
| #764 | 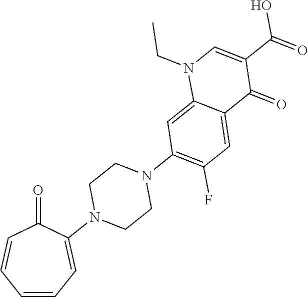 [SMILES: CCn1cc(C(=O)O)c(=O)c2cc(F)c(N3CCN(c4cccccc4=O)CC3)cc21] | 14.5 |
| #765 | 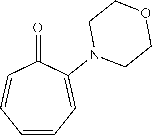 [SMILES: O=c1cccccc1N1CCOCC1] | 24.3 |
| #766 | 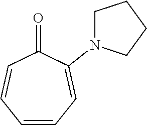 [SMILES: O=c1cccccc1N1CCCC1] | 14 |
| #802 | 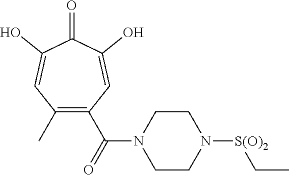 | 0 |
| #838 | 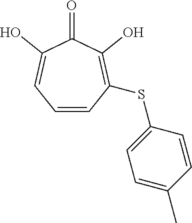 [SMILES: Cc1ccc(Sc2cccc(O)c(=O)c2O)cc1] | 29.3 |
| #840 | 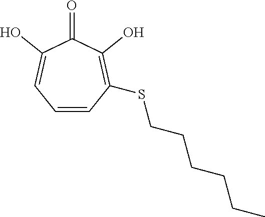 [SMILES: CCCCCCSc1cccc(O)c(=O)c1O] | 74.7 (89.4% on intracellular Mtb) |
| #841 | 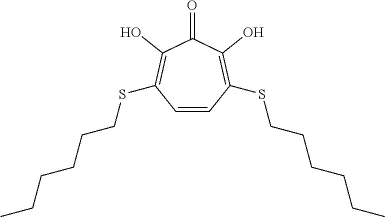 [SMILES: CCCCCCSc1ccc(SCCCCCC)c(O)c(=O)c1O] | 83.5 (91.5% on intracellular Mtb) |
| #842 | 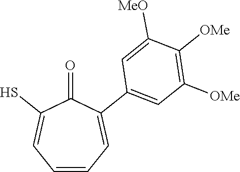 [SMILES: COc1cc(-c2ccccc(S)c2=O)cc(OC)c1OC] | 25 |
| #843 | 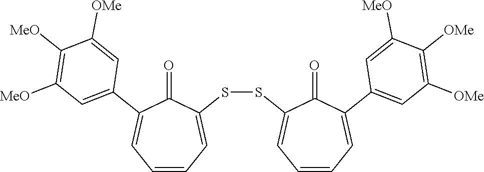 [SMILES: COc1cc(-c2ccccc(SSc3ccccc(-c4cc(OC)c(OC)c(OC)c4)c3=O)c2=O)cc(OC)c1OC] | 26.5 |
| #844 | 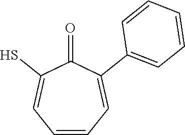 [SMILES: O=c1c(S)ccccc1-c1ccccc1] | 19.1 |
| #845 | 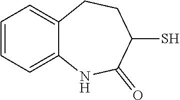 [SMILES: O=C1Nc2ccccc2CCC1S] | 9 |
| #846 | 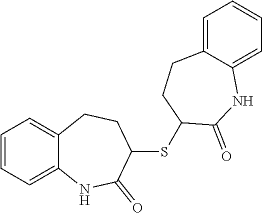 [SMILES: O=C1Nc2ccccc2CCC1SC1CCc2ccccc2NC1=O] | 0 |
| #847 | 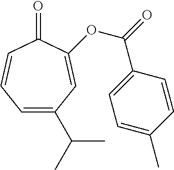 [SMILES: Cc1ccc(C(=O)Oc2cc(C(C)C)cccc2=O)cc1] | 28.8 |
| #859 | 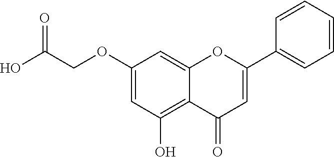 [SMILES: O=C(O)COc1cc(O)c2c(=O)cc(-c3ccccc3)oc2c1] | 11.3 |
| #861 | 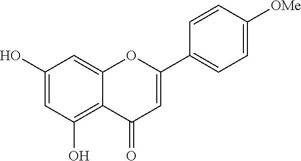 [SMILES: COc1ccc(-c2cc(=O)c3c(O)cc(O)cc3o2)cc1] | 0 |

¹The higher the % inhibition the better the activity on intracellular mycobacteria. All drugs tested at 20 μM.

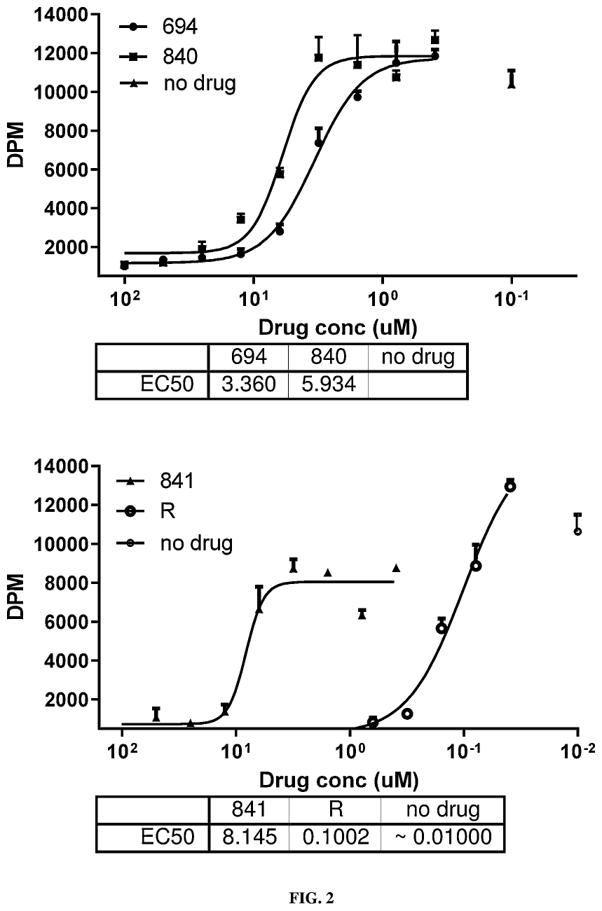

See activity data also shown in FIGS. 1 and 2. The potential for these new troponoids to be developed as anti-mycobacterial drugs was further assessed using ADME prediction model (Qikprop program, version 5.9). The results (Table 2) show that all our new troponoids are predicted to have excellent oral bioavailability and permeation to cells, making them excellent candidates for further development as antimycobacterial drugs.

TABLE 2
In silico ADME prediction

| | | ^{a}Caco-2 | % | | |
|---|---|---|---|---|---|
| | | perme- | human oral | ^{b}Lipiniski | ^{c}Jorgensen |
| Troponoids | MW | ability | bio- | rule of 5 | rule of 3 |
| (ID #) | (daltons) | (nm/sec) | availability | violation | violation |
| 694 | 314.46 | 586 | 96 | 0 | 0 |
| 840 | 226.29 | 596 | 86 | 0 | 0 |
| 841 | 370.56 | 581 | 100 | 0 | 1 |

^{a}Caco-2 cell permeability is a model for the gut-blood barrier and values > 500 nm/sec predict excellent ability to cross the gut-blood barrier.
^{b}For the drug to be orally active in humans, predictions should have no more than 1 violation of the 5 Lipiniski rule criteria: no more than 5 hydrogen bond donors, no more than 10 hydrogen acceptors, molecular mass < 500 daltons, an octanol-water partition coefficient (logP) ≤ 5. The lowest score of 0 is most preferable.
^{c}Jorgensen rule predicts drugs with lead-like properties and criteria include aqueous solubility (Log S > −5.7), Caco2 permeability > 22 nm/sec and number of primary metabolites < 7. The lowest score of 0 is most preferable.

The liver is the principal site of drug metabolism in the body. Therefore, cytochrome P450 (CYP) metabolism is a critically important consideration in the development of drugs for outpatient use and varies widely from one species to another. Interspecies differences in drug metabolism can be predicted by studying the in vitro metabolism of a drug using liver microsomes from different species. Because the murine model will be used for initial drug efficacy studies and our goal is to develop drugs for human use, in vitro liver microsomal studies we performed using both mouse and human liver microsomes. All three compounds (694, 840, and 841) are found to be stable in human and mice liver microsomal studies and confirmed to have good GI absorption by Caco-2 in vitro testing, indicating that all our compounds are excellent candidates for oral administration. Similarly, the pharmacokinetics of four compounds were analyzed and described in Table 3.

TABLE 3
In vitro PK studies: Caco2 and human liver microsome assay

| | | | | | Half-life, |
|---|---|---|---|---|---|
| | | | | | min |
| | | | | | (Human |
| | Caco2 | | | | Liver |
| | PappA > B | PappB > A | Efflux | | Microsome |
| | (sec*cm²) | (sec*cm²) | Ratio | | assay) |
| Verapamil | 4.32E−05 | 1.78E−04 | 4.1 | Ritonavir | <5.0 |
| Warfarin | 9.37E−05 | 2.70E−04 | 2.9 | Warfarin | >30 |
| 284 | 2.67E−06 | 2.53E−06 | 1.0 | 284 | >30 |
| 694 | 5.83E−06 | 1.11E−04 | 19.0 | 694 | 9.8 |
| 840 | 1.45E−05 | 2.11E−04 | 14.6 | 840 | >30 |
| 841 | 3.08E−06 | 5.18E−06 | 1.7 | 841 | 7.0 |

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and/or methods and in the steps or in the sequence of steps of the method described herein without departing from the concept, spirit and scope of the disclosure. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### V. REFERENCES

The following references, to the extent that they provide exemplary procedural or other details supplementary to those set forth herein, are specifically incorporated herein by reference:
Abate et al., “New Verapamil Analogs Inhibit Intracellular Mycobacteria without Affecting the Functions of Mycobacterium-Specific T Cells.” Antimicrob Agents Chemother 60, 1216-1225, doi:10.1128/AAC.01567-15 (2015).
Akers et al., “Thujaplicins from Thuja plicata as iron transport agents for Salmonella typhimurium.” J Bacteriol 141, 164-168 (1980).
Blazevic et al., “Investigations of TB vaccine-induced mucosal protection in mice.” Microbes Infect 16, 73-79, doi:10.1016/j.micinf.2013.09.006 (2014).
Diagnosis and treatment of disease caused by nontuberculous mycobacteria. This official statement of the American Thoracic Society was approved by the Board of Directors, March 1997. Medical Section of the American Lung Association. Am J Respir Crit Care Med 156, S1-25, doi:10.1164/ajrccm.156.2.atsstatement (1997).
Daniel-Wayman et al., “Advancing Translational Science for Pulmonary Nontuberculous Mycobacterial Infections. A Road Map for Research.” Am J Respir Crit Care Med 199, 947-951, doi:10.1164/rccm.201807-1273PP (2019).
Elagawany et al., “Identification of 4-isopropyl—thiotropolone as a novel anti-microbial: regioselective synthesis, NMR characterization, and biological evaluation.” RSC advances 8, 29967 - 29975 (2018).
Eickhoff et al., “Induction of mycobacterial protective immunity by sublingual BCG vaccination.” Vaccine 37, 5364-5370, doi:10.1016/j.vaccine.2019.07.034 (2019).
Ghodousi et al., “Acquisition of Cross-Resistance to Bedaquiline and Clofazimine following Treatment for Tuberculosis in Pakistan.” Antimicrob Agents Chemother 63, doi:10.1128/AAC.00915-19 (2019).
Kumar et al., “Design, Synthesis, and Evaluation of Novel Hybrid Efflux Pump Inhibitors for Use against Mycobacterium tuberculosis. ACS Infect Dis 2, 714-725, doi:10.1021/acsinfecdis.6b00111 (2016).
Kumar et al., “Reversed isoniazids: Design, synthesis and evaluation against Mycobacterium tuberculosis.” Bioorg Med Chem 26, 833-844, doi:10.1016/j.bmc.2017.12.047 (2018).
Meyers et al., “Evaluation of aminohydantoins as a novel class of antimalarial agents.” ACS Med Chem Lett 5, 89-93, doi:10.1021/m1400412x (2014).
Polsfuss et al., “Emergence of Low-level Delamanid and Bedaquiline Resistance During Extremely Drug-resistant Tuberculosis Treatment.” Clin Infect Dis 69, 1229-1231, doi:10.1093/cid/ciz074 (2019).
Zhao, “Plant troponoids: chemistry, biological activity, and biosynthesis.” Curr Med Chem 14, 2597-2621, doi:10.2174/092986707782023253 (2007).

1. A method of treating an infection of a mycobacteria in a patient comprising administering to the patient a therapeutically effective amount of a compound of the formula: 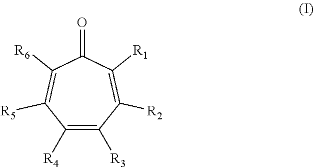
wherein:
R₁ and R₆ are each independently amino, hydroxy, mercapto, alkyl_{(C≤12)}, cycloalkyl_{(C≤12)}, aryl_{(C≤12)}, heteroaryl_{(C≤12)}, heterocycloalkyl_{(C≤12)}, alkoxy_{(C≤12)}, acyloxy_{(C≤12)}, or a substituted version of any of these groups;
R₂ and R₅ are each independently hydrogen or S(O)ₓRₐ, wherein:
x is 0, 1, or 2; and
Rₐ is hydrogen, alkyl_{(C≤12)}, cycloalkyl_{(C≤12)}, aryl_{(C≤12)}, aralkyl_{(C≤12)}, heteroaryl_{(C≤12)}, heterocycloalkyl_{(C≤12)}, or a substituted version thereof; and
R₃ and R₄ are each independently hydrogen, alkyl_{(C≤12)}, substituted alkyl_{(C≤12)}, or —C(O)R_{b}, wherein:
R_{b} is heterocycloalkyl_{(C≤12)}, substituted heterocycloalkyl_{(C≤12)}, heterocycloalkyl_{(C≤12)}-R_{c}, substituted heterocycloalkyl_{(C≤12)}-R_{c}; wherein:
R_{c} is S(O)_{y}R_{c}′, wherein:
y is 0, 1, or 2;
R_{c}′ is hydroxy, alkyl_{(C≤12)}, cycloalkyl_{(C≤12)}, aryl_{(C≤12)}, or a substituted version of any of these groups;
or a compound of the formula: 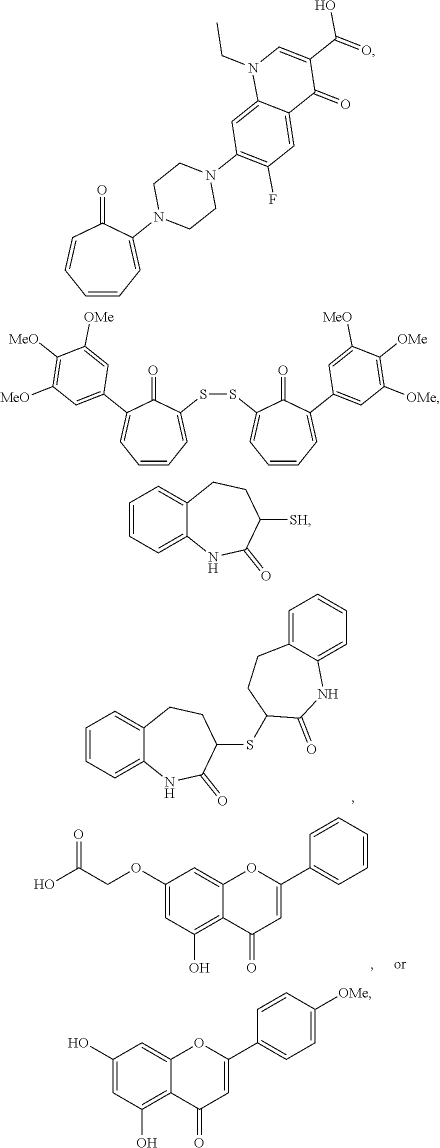
or a pharmaceutically acceptable salt thereof.

2. A method of killing a mycobacteria comprising contacting the mycobacteria with a compound of the formula: 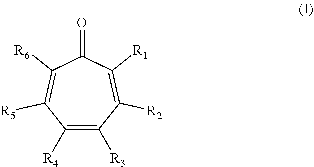
wherein:
R₁ and R₆ are each independently amino, hydroxy, mercapto, alkyl_{(C≤12)}, cycloalkyl_{(C≤12)}, aryl_{(C≤12)}, heteroaryl_{(C≤12)}, heterocycloalkyl_{(C≤12)}, alkoxy_{(C≤12)}, acyloxy_{(C≤12)}, or a substituted version of any of these groups;
R₂ and R₅ are each independently hydrogen or S(O)ₓRₐ, wherein:
x is 0, 1, or 2; and
Rₐ is hydrogen, alkyl_{(C≤12)}, cycloalkyl_{(C≤12)}, aryl_{(C≤12)}, aralkyl_{(C≤12)}, heteroaryl_{(C≤12)}, heterocycloalkyl_{(C≤12)}, or a substituted version thereof; and
R₃ and R₄ are each independently hydrogen, alkyl_{(C≤12)}, substituted alkyl_{(C≤12)}, or —C(O)R_{b}, wherein:
R_{b} is heterocycloalkyl_{(C≤12)}, substituted heterocycloalkyl_{(C≤12)}, heterocycloalkyl_{(C≤12)}-R_{c}, substituted heterocycloalkyl_{(C≤12)}-R_{c}; wherein:
R_{c} is S(O)_{y}R_{c}′, wherein:
y is 0, 1, or 2;
R_{c}′ is hydroxy, alkyl_{(C≤12)}, cycloalkyl_{(C≤12)}, aryl_{(C≤12)}, or a substituted version of any of these groups;
or a compound of the formula: 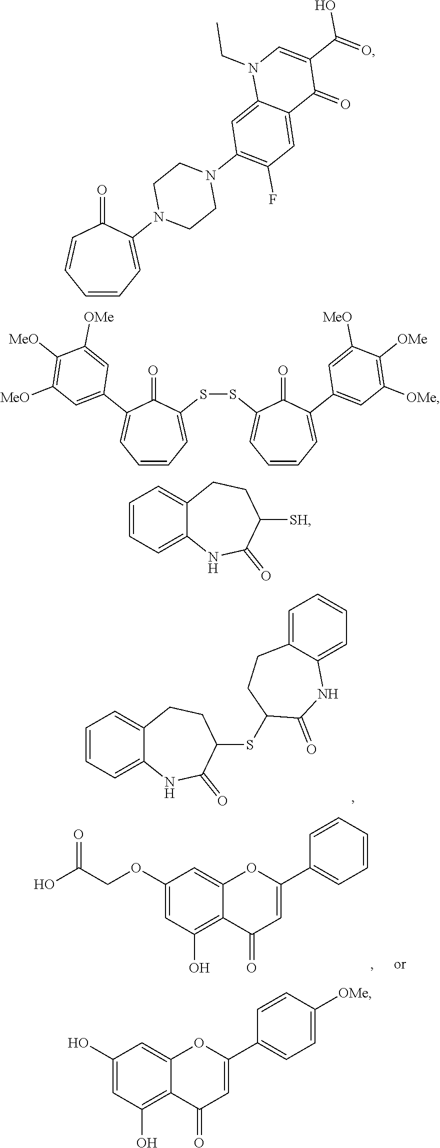
or a pharmaceutically acceptable salt thereof.

3. A method of inhibiting the replication of a mycobacteria comprising contacting the mycobacteria with a compound of the formula: 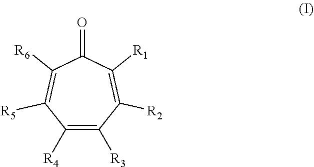
wherein:
R₁ and R₆ are each independently amino, hydroxy, mercapto, alkyl_{(C≤12)}, cycloalkyl_{(C≤12)}, aryl_{(C≤12)}, heteroaryl_{(C≤12)}, heterocycloalkyl_{(C≤12)}, alkoxy_{(C≤12)}, acyloxy_{(C≤12)}, or a substituted version of any of these groups;
R₂ and R₅ are each independently hydrogen or S(O)ₓRₐ, wherein:
x is 0, 1, or 2; and
Rₐ is hydrogen, alkyl_{(C≤12)}, cycloalkyl_{(C≤12)}, aryl_{(C≤12)}, aralkyl_{(C≤12)}, heteroaryl_{(C≤12)}, heterocycloalkyl_{(C≤12)}, or a substituted version thereof; and
R₃ and R₄ are each independently hydrogen, alkyl_{(C≤12)}, substituted alkyl_{(C≤12)}, or —C(O)R_{b}, wherein:
R_{b} is heterocycloalkyl_{(C≤12)}, substituted heterocycloalkyl_{(C≤12)}, heterocycloalkyl_{(C≤12)}-R_{c}, substituted heterocycloalkyl_{(C≤12)}-R_{c}; wherein:
R_{c} is S(O)_{y}R_{c}′, wherein:
y is 0, 1, or 2;
R_{c}′ is hydroxy, alkyl_{(C≤12)}, cycloalkyl_{(C≤12)}, aryl_{(C≤12)}, or a substituted version of any of these groups;
or a compound of the formula: 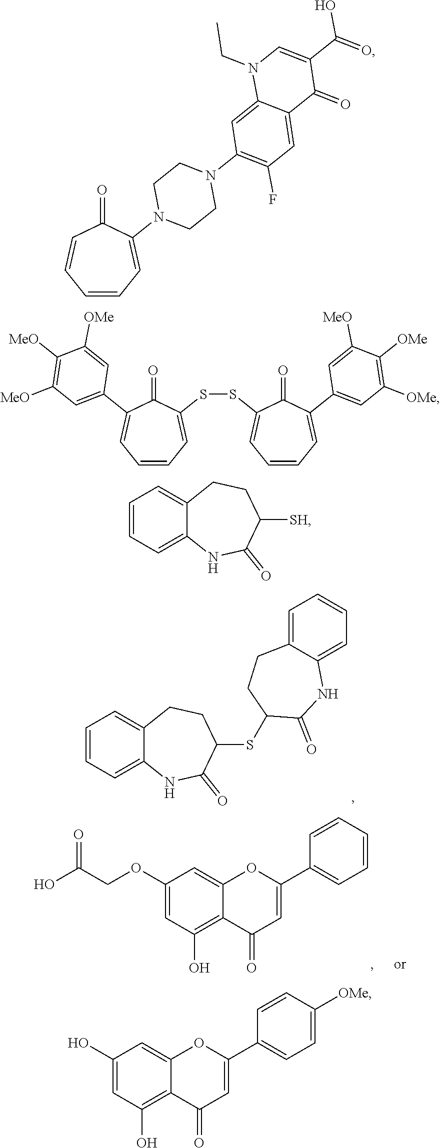
or a pharmaceutically acceptable salt thereof.

4. The method of claim 1, wherein the compound is further defined as: 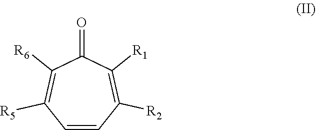
wherein:
R₁ and R₆ are each independently amino, hydroxy, mercapto, alkyl_{(C≤12)}, cycloalkyl_{(C≤12)}, aryl_{(C≤12)}, heteroaryl_{(C≤12)}, heterocycloalkyl_{(C≤12)}, alkoxy_{(C≤12)}, acyloxy_{(C≤12)}, or a substituted version of any of these groups;
R₂ and R₅ are each independently hydrogen or S(O)ₓRₐ, wherein:
x is 0, 1, or 2; and
Rₐ is hydrogen, alkyl_{(C≤12)}, cycloalkyl_{(C≤12)}, aryl_{(C≤12)}, aralkyl_{(C≤12)}, heteroaryl_{(C≤12)}, heterocycloalkyl_{(C≤12)}, or a substituted version thereof; and
or a pharmaceutically acceptable salt thereof.

5. The method of claim 1, wherein the compound is further defined as: 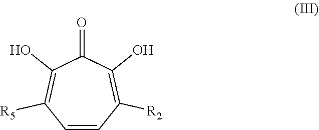
wherein:
R₂ and R₅ are each independently hydrogen or S(O)ₓRₐ, wherein:
x is 0, 1, or 2; and
Rₐ is hydrogen, alkyl_{(C≤12)}, cycloalkyl_{(C≤12)}, aryl_{(C≤12)}, aralkyl_{(C≤12)}, heteroaryl_{(C≤12)}, heterocycloalkyl_{(C≤12)}, or a substituted version thereof; and
or a pharmaceutically acceptable salt thereof.

6. (canceled)

7. The method of claim 1, wherein R₃ is —C(O)R_{b}, wherein: R_{b} is heterocycloalkyl_{(C≤12)}, substituted heterocycloalkyl_{(C≤12)}, heterocycloalkyl_{(C≤12)}-R_{c}, substituted heterocycloalkyl_{(C≤12)}-R_{c}; wherein: R_{c} is S(O)_{y}R_{c}′, wherein: y is 0, 1, or 2; and R_{c}′ is hydroxy, alkyl_{(C≤12)}, cycloalkyl_{(C≤12)}, aryl_{(C≤12)}, or a substituted version of any of these groups.

8-10. (canceled)

11. The method of claim 1, wherein R₁ is heterocycloalkyl_{(C≤12)}or substituted heterocycloalkyl_{(C≤12)}.

12. The method of claim 1, wherein R₁ is aryl_{(C≤12)} or substituted aryl_{(C≤12)}.

13-15. (canceled)

16. The method of claim 1, wherein R₂ is S(O)ₓRₐ, wherein: x is 0, 1, or 2; and Rₐ is hydrogen, alkyl_{(C≤12)}, cycloalkyl_{(C≤12)}, aryl_{(C≤12)}, aralkyl_{(C≤12)}, heteroaryl_{(C≤12)}, heterocycloalkyl_{(C≤12)}, or a substituted version thereof.

17-19. (canceled)

20. The method of claim 16, wherein Rₐ is alkyl_{(C≤12)} or substituted alkyl_{(C≤12)}.

21-22. (canceled)

23. The method of claim 16, wherein Rₐ is aryl_{(C≤12)} or substituted aryl_{(C≤12)}.

24-27. (canceled)

28. The method of claim 1, wherein R₂ is S(O)ₓRₐ, wherein: x is 0, 1, or 2; and Rₐ is hydrogen, alkyl_{(C≤12)}, cycloalkyl_{(C≤12)}, aryl_{(C≤12)}, aralkyl_{(C≤12)}, heteroaryl_{(C≤12)}, heterocycloalkyl_{(C≤12)}, or a substituted version thereof.

29-31. (canceled)

32. The method of claim 28, wherein Rₐ is alkyl_{(C≤12)} or substituted alkyl_{(C≤12)}.

33-34. (canceled)

35. The method of claim 28, wherein Rₐ is aryl_{(C≤12)} or substituted aryl_{(C≤12)}.

36-37. (canceled)

38. The method of claim 1 further defined as: 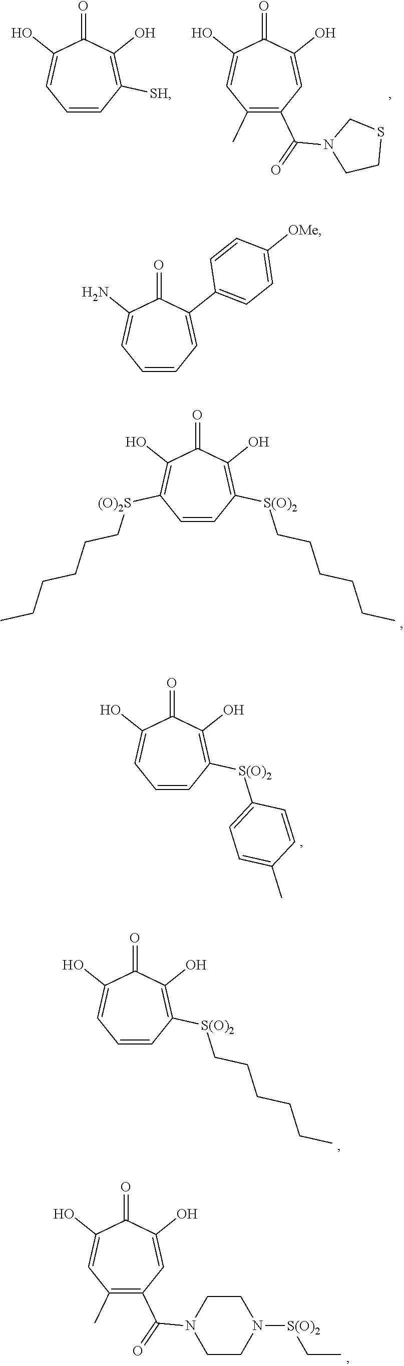 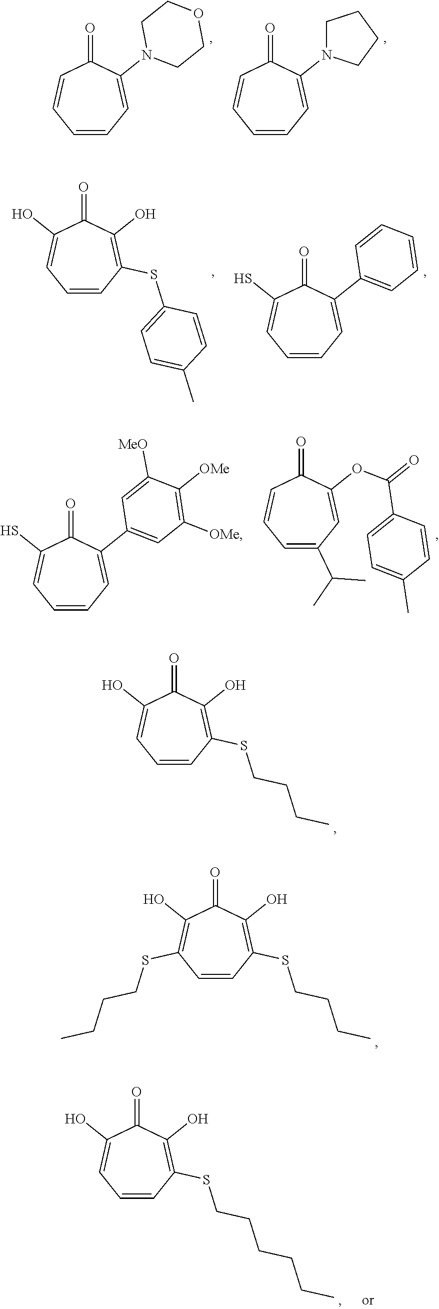 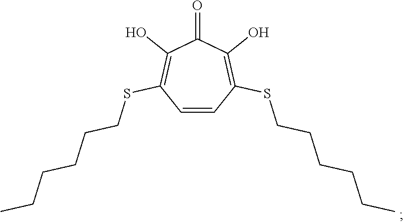 [SMILES: CCCCCCSc1ccc(SCCCCCC)c(O)c(=O)c1O]
or a pharmaceutically acceptable salt thereof.

39. The method of claim 38, wherein the compound is further defined as: 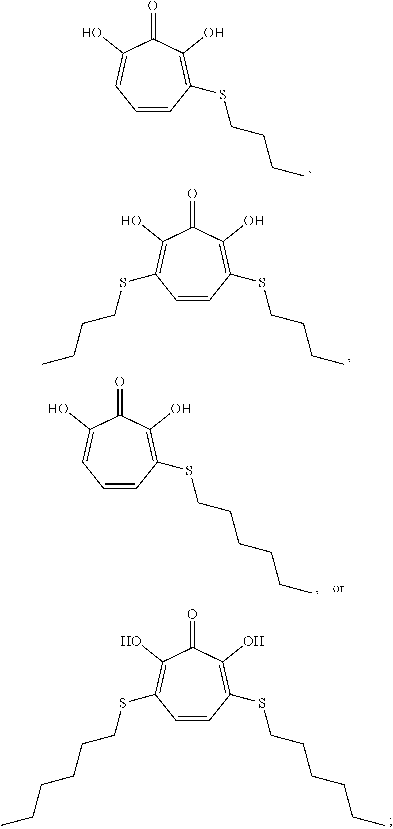
or a pharmaceutically acceptable salt thereof.

40. The method of claim 1, wherein the compound is further defined as: 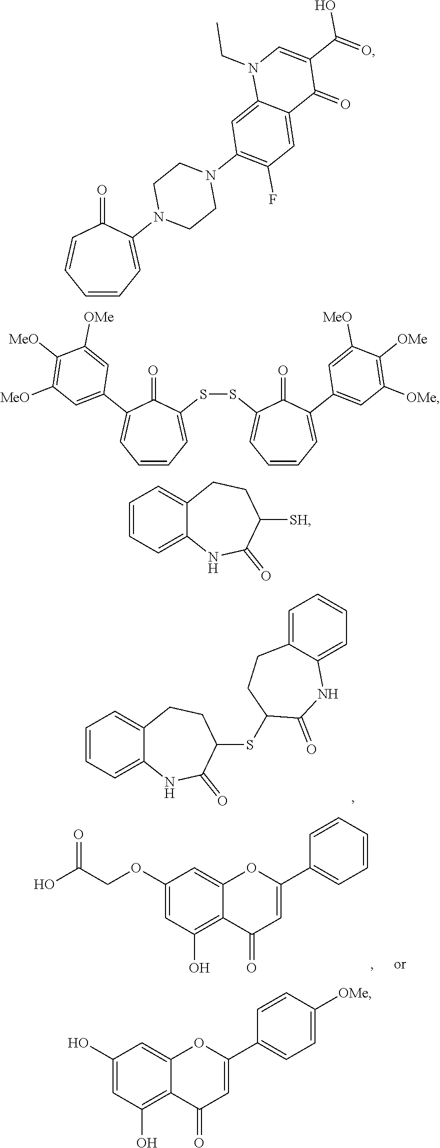
or a pharmaceutically acceptable salt thereof.

41. The method of claim 1, wherein the infection of a mycobacteria is an infection of Mycobacterium tuberculosis.

42-44. (canceled)

45. The method of claim 1, wherein the mycobacteria are resistant to one or more antibiotics.

46-50. (canceled)

51. The method of claim 1, wherein the method further comprises administering a second antibiotic agent.

52-60. (canceled)
